SEQUENCE LISTING (10 sequences)

SEQ ID NO: 1 - DNA, 30 bp, Artificial Sequence
  Forward Primer
ttaattattcatatgtccgcgcccaaaacc

SEQ ID NO: 2 - DNA, 27 bp, Artificial Sequence
  Reverse Primer
aatatactcgagatcgcatccaattgt

SEQ ID NO: 3 - DNA, 30 bp, Artificial Sequence
  Forward Primer
atatccatggcgaaatttaatctgattaaa

SEQ ID NO: 4 - DNA, 27 bp, Artificial Sequence
  Reverse Primer
ataaaaggatccatcgcatccaattgt

SEQ ID NO: 5 - DNA, 906 bp, Yersinia enterocolitica sub. paleartica
cggcgtagaggatcgagatctcgatcccgcgaaattaatacgactcactataggggaattgtgagcggataacaattcccctctagaaataattttgtttaactttaagaaggagatatacatatgtccgcgcccaaaaccatactctcggcacctactgttaccgaacaaaatttgcctgctgcagctattgataactgtcttgtcggctgcccgaccggtggtagtgatcaaaccgttatccgtgatgtgtatacgctgaacaataacagtcataccaaatttgcaaattgggtcgcatataaagtcacaaaaagtagtcaggctagcaaccacccgcgtaaatgggcacaagatcccgacttgccagactcagatacattggcaccggcagattatactggtgctaatcaaaagctggccgttgatcgtgggcatcaggctccgttgagcttactcgctggtaacgaggattcgcaggcacttaattatttgtcaaatatcacaccacaaaaagcggccttaaaccaaggggcatgggtgaggctcgaagatcaagagcgcaatttggctaatcgcccagatgttacggcagtctattccgttaccggccccttatttgaacgccacattgccacactgccagcgaaaccgactgtcgaaatccccagcggatattggaagatcatttttattggcacatcaccggataaaggccagtatgctgcattcctaatggatcaaaatacggccaaatctgctaatttctgcgattatcaagtcaacgttgataccatcgaagcaaaaacaaatccccaattgactatctggtcaaacttgcccgctgatgttgcccaaataattaaatcacagaaagggacgctggctcagacaattggatgcgat

SEQ ID NO: 6 - protein, 284 aa, Yersinia enterocolitica sub. paleartica
MKFNLIKLLPVLLLTACTTTDHSMSAPKTILSAPTVTEQNLPAAAIDNCLVGCPTGGSDQTVIRDVYTLNNNSHTKFANWVAYKVTKSSQASNHPRKWAQDPDLPDSDTLAPADYTGANQKLAVDRGHQAPLSLLAGNEDSQALNYLSNITPQKAALNQGAWVRLEDQERNLANRPDVTAVYSVTGPLFERHIATLPAKPTVEIPSGYWKIIFIGTSPDKGQYAAFLMDQNTAKSANFCDYQVNVDTIEAKTNPQLTIWSNLPADVAQIIKSQKGTLAQTIGCD

SEQ ID NO: 7 - DNA, 837 bp, Yersinia enterocolitica sub. paleartica
atgtccgcgcccaaaaccatactctcggcacctactgttaccgaacaaaatttgcctgctgcagctattgataactgtcttgtcggctgcccgaccggtggtagtgatcaaaccgttatccgtgatgtgtatacgctgaacaataacagtcataccaaatttgcaaattgggtcgcatataaagtcacaaaaagtagtcaggctagcaaccacccgcgtaaatgggcacaagatcccgacttgccagactcagatacattggcaccggcagattatactggtgctaatcaaaagctggccgttgatcgtgggcatcaggctccgttgagcttactcgctggtaacgaggattcgcaggcacttaattatttgtcaaatatcacaccacaaaaagcggccttaaaccaaggggcatgggtgaggctcgaagatcaagagcgcaatttggctaatcgcccagatgttacggcagtctattccgttaccggccccttatttgaacgccacattgccacactgccagcgaaaccgactgtcgaaatccccagcggatattggaagatcatttttattggcacatcaccggataaaggccagtatgctgcattcctaatggatcaaaatacggccaaatctgctaatttctgcgattatcaagtcaacgttgataccatcgaagcaaaaacaaatccccaattgactatctggtcaaacttgcccgctgatgttgcccaaataattaaatcacagaaagggacgctggctcagacaattggatgcgatctcgagcaccaccaccaccaccactgagatccggctgctaacaaagcccgaaag

SEQ ID NO: 8 - protein, 269 aa, Yersinia enterocolitica sub. paleartica
MSAPKTILSAPTVTEQNLPAAAIDNCLVGCPTGGSDQTVIRDVYTLNNNSHTKFANWVAYKVTKSSQASNHPRKWAQDPDLPDSDTLAPADYTGANQKLAVDRGHQAPLSLLAGNEDSQALNYLSNITPQKAALNQGAWVRLEDQERNLANRPDVTAVYSVTGPLFERHIATLPAKPTVEIPSGYWKIIFIGTSPDKGQYAAFLMDQNTAKSANFCDYQVNVDTIEAKTNPQLTIWSNLPADVAQIIKSQKGTLAQTIGCDLEHHHHHH

SEQ ID NO: 9 - DNA, 1070 bp, Yersinia enterocolitica sub. paleartica
cggcgtagaggatcgagatctcgatcccgcgaaattaatacgactcactataggggaattgtgagcggataacaattcccctctagaaataattttgtttaactttaagaaggagatattaccatggcgaaatttaatctgattaaacttttgcctgttcttttgttgaccgcatgtaccacaacagatcactcctccgcgcccaaaaccatactctcggcacctactgttaccgaacaaaatttgcctgctgcagctattgataactgtcttgtcggctgcccgaccggtggtagtgatcaaaccgttatccgtgatgtgtatacgctgaacaataacagtcataccaaatttgcaaattgggtcgcatataaagtcacaaaaagtagtcaggctagcaaccacccgcgtaaatgggcacaagatcccgacttgccagactcagatacattggcaccggcagattatactggtgctaatcaaaagctggccgttgatcgtgggcatcaggctccgttgagcttactcgctggtaacgaggattcgcaggcacttaattatttgtcaaatatcacaccacaaaaagcggccttaaaccaaggggcatgggtgaggctcgaagatcaagagcgcaatttggctaatcgcccagatgttacggcagtctattccgttaccggccccttatttgaacgccacattgccacactgccagcgaaaccgactgtcgaaatccccagcggatattggaagatcatttttattggcacatcaccggataaaggccagtatgctgcattcctaatggatcaaaatacggccaaatctgctaatttctgcgattatcaagtcaacgttgataccatcgaagcaaaaacaaatccccaattgactatctggtcaaacttgcccgctgatgttgcccaaataattaaatcacagaaagggacgctggctcagacaattggatgcgatggatccaattcgagctccgtcgacaagcttgcggccgcactcgagcaccaccaccaccaccactgagatccggctgctaacaaagcccgaaagaa

SEQ ID NO: 10 - protein, 305 aa, Yersinia enterocolitica sub. paleartica
MAKFNLIKLLPVLLLTACTTTDHSSAPKTILSAPTVTEQNLPAAAIDNCLVGCPTGGSDQTVIRDVYTLNNNSHTKFANWVAYKVTKSSQASNHPRKWAQDPDLPDSDTLAPADYTGANQKLAVDRGHQAPLSLLAGNEDSQALNYLSNITPQKAALNQGAWVRLEDQERNLANRPDVTAVYSVTGPLFERHIATLPAKPTVEIPSGYWKIIFIGTSPDKGQYAAFLMDQNTAKSANFCDYQVNVDTIEAKTNPQLTIWSNLPADVAQIIKSQKGTLAQTIGCDGSNSSSVDKLAAALEHHHHHH

(19) United States
(12) Patent Application Publication
(10) Pub. No.: US 2016/0053242 A1
(43) Pub. Date: Feb. 25, 2016

(54) THERMOSTABLE NUCLEASE

(71) Applicant: UNIVERSITE LAVAL, Quebec (CA)

(72) Inventors: Maurice BOISSINOT, Quebec (CA); Karel Boissinot, Quebec (CA); Sandra Isabel, Quebec (CA); Lu-E Shi, Hangzhou (CN)

(21) Appl. No.: 14/867,175

(22) Filed: Sep. 28, 2015

Related U.S. Application Data
(63) Continuation of application No. PCT/CA2014/000156, filed on Feb. 27, 2014.
(60) Provisional application No. 61/770,547, filed on Feb. 28, 2013.

(51) Int. Cl. C12N 9/22; C12P 19/34

(52) U.S. Cl. CPC C12N 9/22; C12P 19/34; C12Y 301/04

(57) ABSTRACT

A heat-stable nuclease found in Y. enterocolitica subsp. Palearctica, named Nucyep, is active in broad spectrum conditions. The gene for Nucyep was sequenced in a strain Y. enterocolitica subsp. palearctica, cloned, and expressed in E. coli, and then purified and characterized. The molecular weight of this enzyme is about 30 to 32 kDa. The translation product, Nucyep1, is biologically active. The purified Nucyep1 exhibits non-specific nuclease activity, being able to degrade various nucleic acids, including RNA, single-stranded DNA (ssDNA) and linear or circular double-stranded DNA (dsDNA). This enzyme is active in a wide range of temperatures, from 0 to 100° C. The enzyme is active in a wide range of pH values from 3.6 to 9.9, and keeps greater than 75% of the activity at pH 7.24. This enterobacterial nuclease has unique levels of intrinsic resistance to heat, and is active under a large spectrum of conditions.

This application is a continuation of International Application No. PCT/CA2014/000156, filed Feb. 27, 2014, now abandoned, which claims the priority of U.S. Provisional Application No. 61/770,547, filed Feb. 28, 2013, the contents of which are herein incorporated by reference in their entirety.

### SEQUENCE LISTING

This instant application contains a Sequence Listing which has been submitted in ASCII format via EFS-Web and is hereby incorporated by reference in its entirety. Said ASCII copy, created on Sep. 28, 2105, is named 8736721860PCTSequenceListing.txt and is 13 Kilobytes (KB) in size.

### FIELD OF THE INVENTION

The present invention relates to a heat-stable (thermostable) nuclease and a gene encoding this enzyme. It also relates to a method for the production of this novel thermostable nuclease using recombinant protein expression techniques. It also relates to a novel thermostable nuclease derived from an organism belonging to Yersinia enterocolitica, more particularly derived from the subspecies palearctica (Nucyep). The invention further relates to a use of Nucyep in reaction for digesting nucleic acids under a wide temperature range and/or extreme pH conditions. The invention further relates to a method for digesting nucleic acids using Nucyep, and a reagent kit to be used in the aforementioned method and use.

### BACKGROUND OF THE INVENTION

### Introduction

Nucleases are enzymes capable of cleaving the phosphodiester bonds between the nucleotide subunits of nucleic acids. Nucleases play very important roles in different aspects of basic genetic mechanisms, including their participation in mutation avoidance, DNA repair, replication and recombination, scavenging of nucleotides and phosphates for the growth and metabolism, host defense against foreign nucleic acid molecules, programmed cell death and establishment of an infection. It has been suggested that nucleases may act as virulence factors. For example, it has been postulated that the nuclease of Vibrio cholerae plays a role during invasion and establishment of an infection and the nuclease from Staphylococcus aureus degrades neutrophil extracellular traps to promote immune cell death. Nucleases are also used in food industry to produce nucleic acid group flavor enhancers, such as guanosine monophosphate and inosine monophosphate, as well as in pharmaceutical industry to produce other nucleotides.

Nucleases are divided into two groups according to substrate specificity: sugar specific nucleases (deoxyribonucleases or ribonucleases) and sugar non-specific nucleases that degrade both DNA and RNA. Among all the non-specific nucleases, the Nuc nuclease of Serratia marcescens has been the most extensively studied^{[4-7]}. This nuclease, commercially available as Benzonase®^{[8]}, is used as a tool in industrial biotechnology for the removal of nucleic acids because of its stability and high catalytic activity.

An alternative nuclease which shows activity at high temperatures with specific or non-specific selectivity for the digestion of nucleic acid molecules may be useful for various applications in the field of molecular biology, for example, SNP analysis, normalization of cDNA library, subtraction and analysis of telomere single strand overhand length and the like. The commercially available Paralithodes camtschaticus nuclease enzyme reagents are purified products from the nature and therefore are considerably expensive. However, although this recombinant nuclease expressed in E. coli is accumulated as an inactive inclusion body in the cell and it can be isolated as an active type enzyme after passing through solubilisation, refolding and the like processes, the procedure requires multiple steps and is therefore complex.

Batzilla, J., Hoper, D., Antonenka, U., Heesemann, J. and Rakin, A. have disclosed the “Complete Genome Sequence of Yersinia enterocolitica subsp. palearctica Serogroup O:3” (J. Bacteriol. 193 (8), 2067 (2011)). In this paper, certain genes were predicted to have nuclease activity, but without addressing any of the surprising/unexpected properties that are disclosed for the nuclease subject of this invention such as resistance at high temperature and nuclease activity under wide temperature and pH ranges.

Under such a background, a novel thermostable nuclease enzyme active under a broad temperature range is desirable. A novel thermostable nuclease which can be easily produced making use of recombinant protein expression techniques is also desirable. Additionally, a novel nuclease having activity over a broad range of pH is also highly desirable.

### SUMMARY OF THE INVENTION

A main aspect intended to be addressed by the present invention is to provide a novel thermostable non-specific nuclease gene and enzyme. Also, there is provided a solution to obtain a novel thermostable nuclease which can be easily produced making use of recombinant protein expression techniques and to provide easy production methods thereof. Additionally, a further aspect is to provide a method for digesting nucleic acid using the novel thermostable nuclease, and a reagent kit to be used in the aforementioned method.

According to a first aspect of the present invention, there is provided an isolated protein comprising, or essentially consisting of, or consisting of: the amino acid sequence represented by SEQ ID NO. 6, 8 or 10, particularly SEQ ID NO. 8. More particularly the protein starts anywhere from amino acid at about position 2 to 20 at its N-terminal and ends anywhere from amino acid at about position 254 to 261 at its C-terminal (as defined according to the numbering in SEQ ID NO.8) so long as the protein has detectable nuclease activity.

Most particularly, the protein comprises at least amino acids 20 to 254 of SEQ ID NO.8. Still, most particularly the protein comprises amino acids 2 to 261 of SEQ ID NO.8. Particularly, this protein is thermostable.

According to a further aspect, the present invention provides a protein as defined herein having nuclease activity within a range of temperature of about 0° C. to about 80° C. and/or having nuclease activity within a range of pH of about 3.6 to about 9.9.

According to a further aspect of the present invention, there is provided a nuclease as defined herein which is obtained from an organism belonging to Yersinia enterocolitica, particularly from Yersinia enterocolitica subsp. palearctica (Yep).

According to a further aspect of the present invention, there is provided a nucleic acid that encodes the protein as defined herein.

According to a further aspect, the present invention provides a composition comprising the protein as defined herein, in admixture with an excipient or in a solution.

According to a further aspect, the present invention provides a method for producing the protein as defined herein, comprising: transforming a host cell with a recombinant vector which comprises a nucleic acid encoding for the protein as defined herein.

According to a further aspect of the present invention, there is provided a method of degrading DNA or RNA, comprising contacting a DNA or RNA molecule with an effective amount of the protein as defined herein under conditions to degrade the DNA or RNA.

According to a further aspect, the present invention provides a method for the production of a flavor-enhancer, comprising contacting a DNA or RNA molecule with an effective amount of the protein as defined herein under conditions to produce 5′ IMP or 5′ GMP.

According to a further aspect, the present invention provides use of the protein as defined herein, for digesting a nucleic acid under heat and/or extreme pH conditions.

According to a further aspect, the present invention provides a plasmid comprising the nucleic acid as defined herein.

According to a further aspect, the present invention provides a cell that is transformed or transduced with the plasmid as defined herein.

According to a further aspect of the present invention, there is provided a kit, which comprises at least one of the protein, or at least one of the nucleic acid, the plasmid, or the cell, all of which as defined herein.

### DETAILED DESCRIPTION OF THE INVENTION

### Description of the Figures

FIG. 1(a) Degradation of PCR products after storage at room temperature for 24 h; FIG. 1(b) Degradation of calf thymus dsDNA by non-heated crude nucleic acids extracts; FIG. 1(c) Degradation of calf thymus dsDNA by heated bacterial cell lysis crude extracts. (See Table 1).

FIG. 2. (a) 2 dimension (2D) gel dyed with SYPRO Ruby; (b) 2D gel dyed with ethidium bromide.

FIG. 3a. Nucleotides and deduced amino-acid sequences of non-specific nuclease from Y. enterocolitica subsp. palearctica CCRI-10035. The predicted signal peptide is italicized; the vertical line between two S's of the amino acid sequence indicates the site of cleavage by the signal peptidase. Asterisk indicates translational stop codon; S.D: Shine-Dalgarno homology; rectangles: the −10 and −35 regions of putative promoter; the active site of non-specific nuclease D-R-G-H is shaded in grey. Converging arrows indicate inverted repeats. Circles: AATAAA supposed to be important to gene regulation.

FIG. 3b. Alignment of DNA/RNA non-specific nuclease. Yep: Yersinia enterocolitica subsp. palearctica CCRI-10035; Yee: Yersinia enterocolitica subsp. enterocolitica 8081 genome sequence; Yf: Yersinia frederiksenii genome project ATCC 33641; Yp: Yersinia pseudotuberculosis IP31758 genome project; ST: Salmonella typhimurium LT2 genome project; Sp: Serratia proteamaculans 568 genome project; Sm: Serratia marcescens genome project. The sequences in bold face mean highly conserved region. The PROSITE motif PDOC00821 is shaded in grey. The sequences underlined for Y. enterocolitica subsp. palearctica is the predicted signal peptide, and for S. marcescens it is the signal peptide.

FIG. 3c. Alignment of amino acids from Nucyep1 with the closest sequences from Y. enterocolitica subsp. enterocolitica (Yee) (these nucleases do not exhibit the same thermal and pH properties). This alignment shows that there is about 94% identity when including the signal peptide (267/283 with Nucyee of the strain WA-314 and 266/283 with Nucyee of the strain 8081). When excluding the signal peptide, there is about 94% identity (244/260) with Nucyee of the strain WA-314 and about 93% identity (243/260) with Nucyee of the strain 8081.

FIG. 4a. SDS-PAGE of Nucyep1 and Nucyep2. 1. Marker 2. Blank vector PET-24a(+) 3. pet24a12 without IPTG induction 4. Supernatant of broth culture after induction 5. PET-24a(+) with IPTG induction 6. pET-24d with IPTG induction 7. Supernatant of broth culture after induction 8. pET-24d without IPTG induction 9. Blank vector pET-24d.

FIG. 4b. NUC1 panel: Degradation of calf thymus dsDNA by Nucyep1. 1. Y. enterocolitica subsp. palearctica CCRI-10035 2. Control blank vector pET24-a+ 3. pET24-a+ with Nucyep1 insert, without IPTG induction, heated at 80° C. for 60 min 4. Nucyep1 without IPTG induction 5. Supernatant of culture broth without IPTG induction 6. Lysate of cells induced by IPTG 7. Lysate of cells induced by IPTG, heated at 80° C. for 60 min 8. Supernatant of culture broth after IPTG induction 9. Supernatant of culture broth heated at 80° C. for 60 min 10. (C) Negative control. 11. Molecular weight markers. NUC2 panel: Degradation of calf thymus dsDNA by Nucyep2. 1. Y. enterocolitica subsp. palearctica CCRI-10035 2. Control blank vector pET24-d+3. pET24-d+ with Nuc2 (heretofore referred as NucYep2) insert, without IPTG induction 4. NucYep1 without IPTG induction, heated at 80° C. for 60 min 5. Supernatant of culture broth without IPTG induction 6. Lysate of cells induced by IPTG 7. Lysate of cells induced by IPTG, heated at 80° C. for 60 min 8. Supernatant of culture broth after IPTG induction 9. Supernatant of culture broth heated at 80° C. for 60 min 10. (C) Negative control. 11. Molecular weight markers

FIG. 5. SDS-PAGE of purified Nucyep1. 1. Cellular extract; 2. Ni-NTA purified Nucyep1; 3. Sample from step 2 purified on Cephadex G-100.

FIG. 6. Ni-NTA purified Nucyep1: Degradation of calf thymus dsDNA at different temperatures.

FIG. 7a. Ni-NTA purified Nucyep1: Degradation of calf thymus dsDNA with different pH value buffer.

FIG. 7b. Ni-NTA purified Nucyep1: Degradation of calf thymus dsDNA after enzyme incubation at different pH for 1 h.

FIG. 8a. Ni-NTA purified Nucyep1: Degradation of calf thymus dsDNA with 2 mM different metal ion.

FIG. 8b. Ni-NTA purified Nucyep1: Degradation of calf thymus dsDNA with 10 mM different metal ion.

FIG. 8c. Ni-NTA purified Nucyep1: Degradation of calf thymus dsDNA with different concentration of CoCl₂, MgCl₂, and MnSO4.

FIG. 8d. Ni-NTA purified Nucyep1: Degradation phenomenon of different substrate with different metal ion. (1-20 mM MgCl₂, 2-20 mM MnSO₄, 3-20 mM CoCl₂, 4—no metal ion, 5—control no enzyme).

FIG. 9a. Ni-NTA purified Nucyep1: Degradation of calf thymus dsDNA by addition of different concentration of Glycerol, BSA, SDS, dNTPs.

FIG. 9b. Ni-NTA purified Nucyep1: Degradation of calf thymus dsDNA by addition of different concentration of EDTA, urea.

FIG. 10. Nucyep1 sequence cloned in expression vector pET24a. Sequence in bold characters represent Nucyep sequence edited to remove the first 23 amino acids (putative signal peptide) of Nucyep and adding the 8 amino acids His-Tag sequence at the carboxyl end, therefore creating the Nucyep1 constructed sequence represented here in capital letters. Underlined characters originate from pET24a vector sequences (initiation codon for methionine (M), and ending with codons for leucine and aspartic acid (L and E) and 6 His-Tag (H)) fused at the carboxyl end of the expressed Nucyep1 nuclease protein. Numbers on the left side correspond to the sequence obtained from transformed plasmid (pET24a vector+Nucyep1 construct) in strain E. coli CCRI-19597.

FIG. 11. Nucyep2 sequence cloned in expression vector pET24d. Sequence in bold characters represent Nucyep sequence containing the 23 amino acids from the signal peptide plus an additional alanine (A) inserted between the first methionine (M) and the lysine (K) for cloning purposes. 13 amino acids originating from the vector are present at the end of the nuclease sequence, but before the 8 amino acids His-Tag sequence at the carboxyl end, therefore creating the Nucyep2 constructed sequence represented here in capital letters. Underlined characters originate from pET24d vector sequences (initiation codon for M, and ending with codons for glycine, serine, asparagine, serine, serine, serine, valine, aspartic acid, lysine, leucine, alanine, alanine, alanine, leucine and aspartic acid (GSNSSSVDKLAAALE) and 6 His-Tag (H)) fused at the carboxyl end of the expressed Nucyep2 nuclease protein. Numbers on the left side correspond to the sequence obtained from transformed plasmid (pET24d vector+Nucyep2 construct) in strain E. coli CCRI-19598.

### ABBREVIATIONS AND DEFINITIONS

### Abbreviations

BSA: bovine serum albumin; dNTP: deoxynucleotide triphosphate; dsDNA: double-stranded deoxyribonucleic acid; ssDNA: single-stranded deoxyribonucleic acid; GST: Gluthation-S-transferase; NTA: nitrilotriacetic acid; Nucyee: nuclease from Yersinia enterocolitica subsp. enterocolitica; Nucyep: thermostable nuclease from Yersinia enterocolitica subsp. palearctica; PAGE: Polyacrylamide gel electrophoresis; RNA: ribonucleic acid; Yee: Yersinia enterocolitica subsp. enterocolitica; Yep: Yersinia enterocolitica subsp. palearctica.

### DEFINITIONS

The term “about” as used herein refers to a margin of + or −10% of the number indicated. For sake of precision, the term about when used in conjunction with, for example: 90% means 90%+1-9% i.e. from 81% to 99%. More precisely, the term about refer to + or −5% of the number indicated, where for example: 90% means 90%+/−4.5% i.e. from 86.5% to 94.5%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The term “hybridization” is to be understood as a bond of a single strand nucleic acid to a complementary or a partially complementary sequence along the lines of the Watson-Crick base pairings in the sample DNA, forming a duplex structure. Particularly, the “stringent hybridization conditions” may comprise 6×SSC, 0.5% SDS, 5×Denhardt and 100 mg/ml of herring sperm DNA at 65° C. for 8 to 16 hours. Alternatively, the “stringent hybridization conditions,” as defined herein, involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N. Y.; and Ausubel et al. (eds.) pages 9.47 to 9.52, 1995 Current Protocols in Molecular Biology, (John Wiley & Sons, N. Y.) at Unit 2.10.

The term “isolated” is used herein to indicate that the protein exists in a physical milieu distinct from that in which it occurs in nature. For example, the isolated protein may be substantially isolated (for example enriched or purified) with respect to the complex cellular milieu in which it naturally occurs, such as in a crude extract. When the isolated protein is enriched or purified, the absolute level of purity is not critical and those skilled in the art can readily determine appropriate levels of purity according to the use to which the material is to be put. In some circumstances, the isolated protein forms part of a composition (for example a more or less crude extract containing many other substances) or buffer system, which may for example contain other components. In other circumstances, the isolated protein may be purified to essential homogeneity, for example as determined spectrophotometrically, by NMR or by chromatography (for example LC-MS).

As used herein, the term “purified” means: at least 90%, for example, 90% or 91% or 92% or 93% or 94% or 95% or 96% or 97% or 98% or 99% or 99.5% or 99.6% or 99.8% or 99.9% or 100% pure.

### DETAILED DESCRIPTION OF PARTICULAR EMBODIMENTS

With the aim of providing an alternative thermostable nuclease, the applicant of the present invention have found that a mRNA encoding for a Nucyep-like polypeptide sequence is expressed in Yersinia enterocolitica subsp. palearctica (Yep). They have isolated a cDNA from said mRNA in the Yep-derived total RNA and thereby revealed that said gene encodes a protein which can actually show the nuclease (Nucyep) activity. By characterizing this protein, it was found to have heat resistance and broad temperature activity range and extreme pH resistance and broad pH activity range. Applicant has provided means to produce this protein making use of a recombinant protein expression technique.

Applicant had observed that PCR amplicons heated (95° C., 5 min) and conserved by freezing DNA crude extracts of Y. enterocolitica subsp. palearctica group I were degraded while stored at 4° C. prior to gel analysis. It was therefore thought that the Y. enterocolitica subsp. palearctica DNA crude extracts may contain a heat-stable nuclease, resistant to freezing and active at 4° C. in standard PCR reaction buffer.

Nakajima et al.^{[12]} had already noticed some PCR products degradation phenomenon in Y. enterocolitica strains. Among all the Yersinia strains studied by the Applicant, Y. enterocolitica subsp. palearctica strain CCRI-10035 presented the strongest heat-stable nuclease activity.

### The Following Describes the Present Invention in Detail.

### Thermal Resistance of the Protein (Pre-Treatment)

The nuclease of the present invention is resistant to exposure at high temperatures prior to digestion of nucleic acids. Exposure of the nuclease to 80° C. for 40 minutes resulted in 20% to 50% of the activity of non-heated nuclease for digestion of calf thymus dsDNA at 37° C. Exposure of the nuclease to 80° C. for 60 minutes or 100° C. for 20 minutes resulted in ˜5% of the activity of non-heated nuclease for digestion of calf thymus dsDNA at 37° C. Exposure to longer times at 100° C. resulted in undistinguishable results when compared to undigested controls (i.e. no remaining nuclease activity).

### Heat Stability of Nuclease Activity

The thermostable Nucyep of the present invention has such stability and activity at a broad range of temperatures that it can show nuclease activity at least within a range of from about 0° C. to about 100° C., particularly high activity within a range of from about 20° C. to about 60° C., and particularly optimum activity at about 35° C. Additionally, another aspect of the heat resistance possessed by the enzyme of the present invention is such that it can keep at least about 80%, particularly about 90%, most particularly 95% of its activity while it is being heated or after having been heated.

Although the thermostable Nucyep of the present invention can show particularly high activity within a range of from about 35° C. to about 40° C. as described herein, it can show a sufficient activity even at a low temperature (e.g., 0° C.). Namely, the thermostable Nucyep of the present invention can be used within a broad temperature range of from a low temperature of 0° C. to a high temperature of about 80° C. Additionally, when partial inactivation of the enzyme is not a problem, it can also be used at a higher temperature within such a range that the activity of this enzyme does not completely disappear (e.g., a temperature exceeding 80° C. for a limited time such as for example 100° C.).

The thermostable Nucyep of the present invention can show sufficient activity even at low temperatures (e.g., 4° C.). Since the thermostable Nucyep of the present invention can show its activity within a broad temperature range from a low temperature to a high temperature, those skilled in the art can use it under various temperature conditions in function of the intended purpose. For example, the nucleic acid digestion method of the present invention can be carried out by using the thermostable Nucyep of the present invention under constant temperature conditions, under varying temperature conditions (within a certain range), or under predetermined or programmed temperature cycling conditions and the like.

Particularly, the protease has detectable nuclease activity within a wide range of temperatures such as for example, with a lower limit from about 0° C., or about 5° C., or about 10° C., or about 15° C., or about 20° C.; with an upper limit of about 60° C. or about 65° C. or about 70° C. or about 75° C. or about 80° C. Particularly, the nuclease is active from about 0° C. to about 80° C.; from about 4° C. to about 70° C.; from about 10° C. to about 60° C.; from about 15° C. to about 50° C.; and from about 20° C. to about 40° C.

Yersinia enterocolitica subsp. palearctica

Yersinia enterocolitica is a zoonotic enteropathogen that encompasses strains of varying pathogenicity and tremendous genetic diversity. Pathogenic Y. enterocolitica strains cause acute gastroenteritis, which are usually self-limited but may result in severe post-infectious complications. Neubauer et al. have separated Y. enterocolitica strains into two subspecies based on 16S RNA sequences and lysine decarboxylase activity: Y. enterocolitica subsp. enterocolitica and Y. enterocolitica subsp. palearctica. Isabel et al. studied the phylogeny of Y. enterocolitica strains based on tuf gene^{[11]}. Y. enterocolitica subsp. enterocolitica were included in one clade while Y. enterocolitica subsp. palearctica strains were separated into two subgroups^{[11]}.

The thermostable Nucyep of the present invention is a Nucyep derived from an organism belonging to Yersinia, more illustratively derived from an organism belonging to Yersinia enterocolitica, further illustratively derived from the subspecies palearctica. Particularly, the thermostable Nucyep of the present invention is a Nucyep derived from an organism belonging to a strain of Yersinia enterocolitica subsp. palearctica (Yep) selected from the group consisting of: CCRI-10035, CCUG 4586 (CCRI-14538), CCUG 21476 (CCRI-14540) and CCUG 31436 (CCRI-14542).

### Sequence

One particular embodiment of the thermostable Nucyep of the present invention is a Nucyep comprising an amino acid sequence of the Yersinia enterocolitica subsp. palearctica. In a particular case, the Nucyep protein comprises, essentially consists of, or consists of an amino acid sequence represented by SEQ IDs NO. 6, 8 or 10, and more particularly amino acids 2 to 261 of SEQ ID NO. 8. In an alternative embodiment, the Nucyep protein has an amino acid sequence in which one or two or more amino acids of said amino acid sequence are added, deleted, inserted or substituted. In a particular embodiment the Nucyep protein has an amino acid sequence in which up to its first 24 amino acids (corresponding of the signal peptide+Met) have been deleted of SEQ IDs NO. 6 or 10. In a particular embodiment the Nucyep protein has an amino acid sequence in which up to eight (8) C-terminal amino acids (His tags) are deleted from SEQ IDs NO. 8 or 10.

In particular, the Nucyep protein comprises, essentially consists of, or consists of the amino acid sequence represented by SEQ ID NO. 6, 8 or 10 in which one or two amino acids are substituted. More particularly, the Nucyep protein comprises, essentially consists of, or consists of the amino acid sequence represented by SEQ ID NO. 6, 8 or 10 in which one to ten amino acids are added. Still, particularly, the Nucyep protein comprises, essentially consists of, or consists of the amino acid sequence represented by SEQ ID NO. 6, 8 or 10 in which one or two amino acids are deleted. Most particularly, the Nucyep protein comprises, essentially consists of, or consists of the amino acid sequence represented by SEQ ID NO. 6, 8 or 10 in which one or two amino acids are inserted.

In particular, the molecular weight of the thermostable Nucyep of the present invention is about 29,000 Daltons to about 32,000 Daltons when measured by SDS polyacrylamide gel electrophoresis. Also, particularly, the isoelectric point of the thermostable Nucyep of the present invention is about 5.9.

In this case, the amino acid sequence in which one or two or more amino acids are added, deleted, inserted or substituted (whether naturally or synthetically), means an amino acid sequence having an amino acid sequence identity of at least 95%, preferably at least 96%, more preferably at least 97%, further preferably at least 98%, most preferably at least 99%, in comparison with a basic sequence of SEQ ID NO. 6, 8 or 10.

Particularly, the amino acid sequence in which one or two or more amino acids are added, deleted, inserted or substituted (whether naturally or synthetically) means, an amino acid sequence having an amino acid sequence identity of at least 95%, preferably at least 96%, more preferably at least 97%, further preferably at least 98%, most preferably at least 99%, in comparison with amino acids 2 to 261 of SEQ ID NO. 8.

Predictably, the nuclease can retain its activity when the protein is partially cleaved or mutated either as its N-terminal or C-terminal without affecting amino acids situated at, or near, the enzyme's active site or contributing to the protein 3D-structure stability. In the present case, based on 3D-structure analysis, amino acid from position 2 to about position 20 at the N-terminal and amino acids from position about 254 to position 261 may be deleted, in whole or in part, without completely destroying the nuclease activity of the cleaved protein.

Most particularly the protein may start anywhere from amino acid at position 2 to 20 (i.e. 2, 3, 4, 5, 6, 7, 8, 9, 10, 11, 12, 13, 14, 15, 16, 17, 18, 19 or 20) at its N-terminal and may end anywhere from amino acid at position 254 to 261 (i.e. 254, 255, 256, 257, 258, 259, 260 or 261) at its C-terminal (as defined according to the numbering in SEQ ID NO. 8), so long as the protein has detectable nuclease activity.

### Nuclease Substrate

According to the present invention, the Nucyep means an enzyme which has the ability to show a non-specific Nucyep activity, and said Nucyep activity means an activity to preferentially digest or degrade deoxyribonucleic acid existing in linear, circular, single-stranded or double-stranded nucleic acids. An RNA, a DNA single strand, a DNA-DNA double strand, a DNA-RNA double strand and a double strand moiety in a nucleic acid molecule having a partial single strand structure and a partial double strand structure are included in said nucleic acid. Said partial single strand structure includes, for example, mismatch of bases, bulge structure, loop structure, flap structure and pseudo-Y structure and the like.

The Nucyep nuclease activity can be measured for example by the Kunitz method (Kunitz M. 1950, J. Gen. Physiol., 33, 349-362). Additionally, the Nucyep nuclease activity can also be detected for example by allowing the purified enzyme to undergo the reaction at 37° C. for 30 minutes using a nucleic acid as the substrate, carrying out an agarose gel electrophoresis and comparing the presence or absence of the intact substrate band.

### Co-Factors

In particular, the thermostable Nucyep of the present invention shows suitable Nucyep1 nuclease activity in the presence of Co²⁺ ion, Mn²⁺ ion or Mg²⁺ ion. Still, particularly, the thermostable Nucyep of the present invention is inhibited by the presence of Zn²⁺, Ca²⁺, Cu²⁺, EDTA or SDS. More particularly, the thermostable Nucyep of the present invention is inhibited by the presence of 2 mM: Zn²⁺, Ca²⁺, or Cu²⁺; 100 mM EDTA or 0.1% SDS.

An example of the utility of these co-factors is the fact that the Nucyep activity is able to degrade a linear double-stranded DNA or a single-stranded DNA without the addition of divalent metal cation mentioned above, but not the circular dsDNA or RNA. However, calf thymus dsDNA can contribute residual ions resulting in limited nuclease activity.

### Method of Isolation

A preferable embodiment of the thermostable Nucyep of the present invention is an enzyme purified from Yersinia enterocolitica, subsp. palearctica (Yep). Based on the disclosure of the present invention that Yep is expressing a thermostable Nucyep1, those skilled in the art can isolate and purify the thermostable Nucyep1 of the present invention from Yep, for example, can be prepared as a recombinant protein making use of a gene encoding for the thermostable Nucyep1 of the present invention.

The gene encoding for the thermostable Nucyep of the present invention is prepared by a conventionally known method such as a cloning technique and the like to those skilled in the art, based on SEQ ID NO. 5, 7 or 9 of this specification. For example, the gene can be prepared as a cDNA from total RNA derived from Yep. Additionally, the gene is prepared, for example, by the method described in Example 1 of this specification based on the disclosed sequences.

Additionally, the aforementioned DNA having a nucleotide sequence encoding for the amino acid sequence of the thermostable Nucyep is also included in the gene encoding for the thermostable Nucyep1 of the present invention. Also, a DNA which hybridizes with said DNA or a DNA consisting of a sequence complementary to said DNA under a stringent condition and which encodes a protein having the Nucyep1 activity is also included in the gene of the present invention. Additionally, a DNA which has a nucleotide sequence in which one or two or more bases in a DNA having a nucleotide sequence encoding for the thermostable Nucyep described in SEQ ID NO. 6, 8 or 10 are added, deleted or substituted, and which also has a nucleotide sequence encoding for the thermostable Nucyep, is also included in the gene of the present invention. In this case, the nucleotide sequence in which one or two or more bases are added, deleted, or substituted means a nucleotide sequence having a nucleotide sequence homology of at least 75%, preferably at least 80%, more preferably at least 85%, further preferably at least 90%, most preferably at least 95%, in comparison with the basic sequence of SEQ ID NO. 5, 7 or 9.

In this case, the “stringent condition” means a condition under which a so-called specific hybrid is formed and non-specific hybrids are not formed. Although it is difficult to express this condition numerically and distinctly, for example, it is a condition under which a pair of nucleic acids having high homology, such as a pair of DNA having 80% or more, preferably 85% or more of homology, are hybridized but other nucleic acids having homology of lower than that are not hybridized. Namely, examples of the stringent conditions include a condition for effecting hybridization by incubating a solution containing 6×SSC (composition of 1×SSC: 0.15 M sodium chloride and 0.015 M sodium citrate, pH 7.0), 0.5% SDS, 5×Denhardt and 100 mg/ml of herring sperm DNA at 65° C. for a period of from 8 to 16 hours together with a probe, and the like can be exemplified.

Also, in addition to the method for preparing a thermostable Nucyep from a cell-derived material, a desired thermostable Nucyep gene can be directly synthesized by an organic synthesis method, an enzymatic synthesis method or an optionally combined method thereof, based on the confirmed thermostable Nucyep nucleotide sequence information as defined in SEQ ID NO. 5, 7 or 9.

The recombinant DNA of the present invention can be obtained by connecting the gene of the present invention to (inserting into) an appropriate vector. The vector into which the gene of the present invention is inserted is not particularly limited with the proviso that it is replicable in its host, and examples thereof include a plasmid DNA, a phage DNA and the like can be exemplified. Examples of the plasmid DNA include plasmids derived from Escherichia coli (e.g., pBR322, pBR325, pUC8, pUC9, pUC118, pUC119, pET (manufactured by Novagen), pGEX (manufactured by Amersham Biosciences), pQE (manufactured by QIAGEN), pMAL (manufactured by New England Biolabs) and the like), plasmids derived from Bacillus subtilis (e.g., pUB110, pTP5 and the like), plasmids derived from yeast (e.g., YEp13, YEp24, YCp50 and the like) and the like. Examples of the phage DNA include lambda phage and the like. In addition to these, the examples include retrovirus, vaccinia virus and the like animal virus vectors and baculovirus and the like insect virus vectors.

For inserting the gene of the present invention into a vector, for example, a method in which a DNA containing purified gene of the present invention is digested with appropriate restriction enzymes, inserted into restriction enzyme sites or a multi-cloning site of an appropriate vector DNA and thereby connected to the vector, or the like is employed. In addition to a promoter and the gene of the present invention, an enhancer or the like cis element, a splicing signal, a poly(A) addition signal, a selection marker, a ribosome binding sequence (Shine Dalgarno) and the like can be connected to the vector as occasion demands. Examples of the selection marker include a dihydrofolate reductase gene, an ampicillin resistance gene, a neomycin resistance gene and the like. In order to facilitate purification and detection of the thermostable Nucyep of the present invention later, or in order to prevent insolubilization of the expressed thermostable Nucyep1 in cells, a sequence which encodes GST tag, histidine tag or the like tag sequence and the like may be added to the gene of the present invention (e.g., Appl. Microbiol. Biotechnol., 60, 523-533, 2003).

More particularly, the invention comprises an expression vector wherein the Nucyep1 gene has been inserted. Most particularly, this expression vector is a plasmid for expression in a cell transformed or transduced therewith. More particularly, the expression vector is a plasmid identified as pET-Nucyep1 such as the one described in Example 1.7 and/or deposited at the International Depository of Canada (IDAC) on Feb. 13, 2013 under accession number IDAC 130213-03.

The transformed cell and transduced cell of the present invention can be obtained by introducing the recombinant vector of the present invention into a host in such a manner that the gene of interest can be expressed. The host to be used herein is not particularly limited with the proviso that it can express the gene of the present invention. Example thereof include, bacteria belonging to the genus Escherichia (Escherichia coli or the like), the genus Pseudomonas (Pseudomonas putida or the like), the genus Bacillus (Bacillus subtilis or the like), the Rhizobium (Rhizobium meliloti or the like) and the like, Saccharomyces cerevisiae, Schizosaccharomyces pombe and the like yeasts, COS cell, CHO cell and the like animal cells and an army worm cell (Sf9, Sf21 or the like) and a silkworm cell (BmN4 or the like) and the like insect cells.

When the transformed cell or transduced cell of the present invention is obtained, enzyme collection means the collection generally carried out by those skilled in the art can be used for collecting the thermostable Nucyep of the present invention from its cultured product. When the thermostable Nucyep of the present invention is produced in a microbial body or cell, said enzyme can be extracted by disintegrating the microbial body or cell. For example, said enzyme can be extracted from the microbial body or cell by a method in which a microbial body or cell is subjected to an ultrasonic disintegration treatment, French press cell lysis, grinding treatment or the like in the usual way, a method in which said enzyme is extracted using lysozyme or the like lytic enzyme, or a method in which said enzyme is discharged into outside moiety of the microbial body or cell by shaking it or leaving it in the presence of solvent or the like to cause its autolysis. Alternatively, the thermostable Nucyep of the present invention can be secreted from microbial body or cell, for example using secretion expression vector as previously described (Boissinot M et al 1997, EMBO J. 16:2171-8),

When a purified enzyme preparation is obtained from the thus obtained preparation containing the thermostable Nucyep of the present invention, by further purifying the thermostable Nucyep of the present invention, the thermostable Nucyep of the present invention can be isolated and purified from the aforementioned enzyme solution by carrying out general biochemical methods used by those skilled in the art for the isolation and purification of protein, such as ammonium sulfate precipitation, gel chromatography, ion exchange chromatography, affinity chromatography and the like, alone or in an optional combination.

When the thermostable Nucyep of the present invention is expressed by adding GST tag, histidine tag or the like tag sequence and the like thereto, such an addition sequence may be removed during purification or after purification of said enzyme by an appropriate enzyme treatment generally carried out by those skilled in the art, or the like, or may be used as such when activity of the thermostable Nucyep of the present invention is not spoiled by the addition sequence.

An example of the particular embodiment of the method for preparing the thermostable Nucyep of the present invention as a recombinant protein is a method for expressing the protein of interest in a bacteria or any prokaryotic cell, a eukaryotic cell, a preferable example thereof is a method for expressing the protein of interest in an insect cell, and for example, a method which uses a baculovirus-insect cell recombinant protein expression system in which a protein of interest is expressed in a recombinant baculovirus-infected insect cell can be cited.

An example of the particular embodiment of the method for producing the thermostable Nucyep of the present invention, which uses E. coli bacteria recombinant protein expression system, is described illustratively and further in detail in the examples of this specification. More particularly, the expressed His-tagged recombinant protein was isolated from the crude cell extract and the recombinant protein and purified from elution fractions collected from a Ni-NTA agarose column.

### Uses and Methods of Use

By the use of the thermostable Nucyep of the present invention, a method for digesting a nucleic acid using this enzyme is provided. According to this method, a nucleic acid can be digested non-specifically. More in detail, according to this method, a linear double-stranded DNA or single-stranded DNA can be preferentially degraded rather than a circular double-stranded DNA or RNA in a system in which the linear and circular double-stranded DNA coexist. When a linear DNA is specifically digested using the thermostable Nucyep of the present invention, it is preferable to carry out the reaction without the addition of Co²⁺, Mg²⁺ or Mn²⁺ ions.

Particularly, the Nucyep of the present invention can be used for nucleic acids production. More the Nucyep of the present invention can be used for the degradation of nucleic acid contaminating products in the biotechnological industry. More particularly, the Nucyep1 can be used to degrade contaminating nucleic acids resulting from the production of vaccines, recombinant proteins and PCR reagents. Still, more particularly, the Nucyep1 of the present invention can be used for the production of flavor-enhancers such as 5′ IMP and 5′ GMP. The Nucyep of the present invention can be used to degrade nucleic acids for signal generation, to degrade nucleic acids for step in a nucleic acid amplification method, etc.

The reaction liquid composition and reaction conditions in the nucleic acid digestion method which uses the thermostable Nucyep of the present invention can be optionally selected by those skilled in the art in function of the purpose. For example, the reaction liquid composition and reaction conditions described in the examples of this specification and modifications thereof can be used, although not limited thereto. As described in the foregoing, although the thermostable Nucyep of the present invention can show particularly high activity within the range of from about 35° C. to about 40° C., the applicable reaction temperature is not limited to this range.

### Kits

A reagent kit which comprises the thermostable Nucyep of the present invention is also included in the scope of the present invention. This reagent kit can contain substances which are necessary for carrying out the measurement such as a marker, a buffer solution, a salt and the like. In addition, it may contain substances such as a stabilizer and/or an antiseptic and the like. It is possible to use said reagent kit in the non-specific nucleic acid digestion method of the present invention.

By the present invention, there is provided a novel Nucyep which has the Nucyep activity and has such a heat resistance that it can show activity within a range of at least from about 0° C. to 100° C., particularly can show particular activity within a range of from about 4° C. to 80° C., more particularly can show particularly high activity within a range of from about 20° C. to 60° C., most particularly preferably can show optimum activity at about 35 to 40° C., or has such a heat resistance that, after heating at 80° C. for 40 minutes, it can keep about 50% to about 80% of its original activity (i.e. before heating).

There is also provided a gene encoding for the thermostable Nucyep, a recombinant DNA containing the gene, a transformed cell or transduced cell containing the recombinant DNA, and a method for producing said thermostable Nucyep. Additionally, a method for digesting nucleic acid using the thermostable Nucyep and a reagent kit to be used in the aforementioned methods are provided by the present invention. The thermostable Nucyep of the present invention can be applied to various inspection techniques, diagnostic techniques and genetic engineering techniques, which include analysis, detection, degradation, synthesis, modification, amplification, sequencing, and the like of nucleic acid molecules.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

This disclosure describes the cloning and sequence determination of the gene coding the heat-stable non-specific nuclease from Y. enterocolitica subsp. palearctica CCRI-10035. This gene was overexpressed in E. coli and the His-tagged recombinant protein was purified by Ni-NTA column. Its characterization was partially investigated.

### Example 1

### Materials and Methods

### 1.1 Preparation of Crude Nucleic Acids Extracts

Crude nucleic acids extracts were prepared by the IDI Lysis kit as described (Ke et al 2000, Clin Chem. 46:324-31, Aldous et al., 2005 J Clin Microbiol. 43:2471-3). Briefly, bacterial strains (see Table 1) were grown at 30° C. or 37° C. under aerobic conditions on trypticase soy agar medium (TSA) with 5% sheep blood overnight. Cells were resuspended in phosphate buffer saline (PBS) and adjusted to a 0.5 McFarland standard (Fisher Scientific Company, Ottawa, Ontario, Canada) using a nephelometer. A 100 μl bacterial suspension was added into 1.5 ml microtube containing the IDI Lysis mix of glass beads, centrifuged at 10 000 g for 1 min. Supernatant was discarded. Then, 100 μl of TE (Tris EDTA, pH 8.0) 5× was added into the above tube. The mixtures were vortexed for 5 min, heated at 95° C. for 5 min, and kept frozen at −20° C. prior to PCR.

### 1.2 PCR Amplicon Degradation Phenomenon

E. coli ATCC 11775^{T} (CCRI-467), Hafnia alvei ATCC 13337^{T} (CCRI-545), Klebsiella pneumoniae ATCC 13883^{T} (CCRI-566), Obesumbacterium proteus ATCC 12841^{T} (CCRI-642), Salmonella enterica subsp. enterica ATCC 13076^{T} (CCRI-722), Salmonella enterica subsp. enterica serovar Typhimurium ATCC 14028 (CCRI-717), Serratia marcescens ATCC 13880^{T}(CCRI-736), Staphylococcus aureus ATCC 43300 (CCRI-175), Yersinia aldovae ATCC 35236^{T} (CCRI-14535), Yersinia aleksiciae LMG 22254^{T} (CCRI-15962), Yersinia bercovieri ATCC 43970^{T} (CCRI-763), Y. enterocolitica subsp. enterocolitica ATCC 9610^{T}(CCRI-764), ATCC 27729 (CCRI-2161), CCUG 8238 (CCRI-14541), Y. enterocolitica subsp. palearctica CCRI-10035, CCUG 4586 (CCRI-14538), CCUG 18381 (CCRI-14539), CCUG 21476 (CCRI-14540) and CCUG 31436 (CCRI-14542), Yersinia frederiksenii ATCC 33641^{T} (CCRI-765) and ATCC 29912 (CCRI-14530), Yersinia intermedia ATCC 29909^{T} (CCRI-766), Yersinia kristensenii ATCC 33638^{T} (CCRI-767), Yersinia mollaretii ATCC 43696^{T} (CCRI-768), Yersinia pseudotuberculosis ATCC 29833^{T} (CCRI-769), Yersinia rohdei ATCC 43380^{T} (CCRI-771), Yersinia ruckeri CCRI-10643 and Yokenella regensburgei ATCC 35313^{T} (CCRI-574).

The presence of thermostable nuclease activity in representative Yersinia, other Enterobacteriaceae as well as one S. aureus strains was evaluated. PCR mixtures contained primers to amplify 16S rDNA amplicons each at 1.0 μM, 200 μM deoxyribonucleoside triphosphates (Amersham Biosciences), 10 mM Tris-HCl (pH 9.0), 50 mM KCl, 0.1% Triton X-100, 2.5 mM MgCl₂, 3.3 μg/μl bovine serum albumin (BSA) (Sigma-Aldrich Canada Ltd.), and 0.025 U/μL Taq DNA polymerase (Promega). Crude DNA (see Table 1) extracts (1 μl) was added to each PCR reaction (19 μl), which was subjected to thermal cycling (3 min at 94° C. followed by 40 cycles of 1 s at 95° C., 1 min at 60° C., and 1 min at 72° C., with a final extension of 7 min at 72° C.) using a PTC-200 DNA Engine thermocycler (Bio-Rad Laboratories,). Amplicons were detected using 1.2% agarose gel electrophoresis with 0.25 μg/mL of ethidium bromide in Tris-borate-EDTA buffer (89 mM Tris, 89 mM boric acid, 2 mM EDTA). Amplicons' degradation phenomenon was verified with a 1 kb DNA ladder used as a molecular weight marker (Invitrogen). An amplicon produced from nuclease-free purified genomic DNA prepared as described (Ke et al 200, Clin Chem. 46:324-31), was used as negative control.

### 1.3 SDS-PAGE Electrophoresis

The Laemmli buffer system was applied with a vertical Bio-Rad mini-gel apparatus. After electrophoresis, protein bands were stained using Coomassie Brilliant Blue R-250 (0.2%) in 25% methanol and 10% acetic acid, and then destained with warm water.

### 1.4 Active Two-Dimensional Gel Electrophoresis

Protein concentration was measured by the method of Bradford. An 18 cm Immobiline™ pH 4-7 DryStrip was applied. Proteins (200 μg) were diluted in T8 buffer (7 M Urea, 3% W/V CHAPS; 3-[(3-cholamidopropyl)dimethylammonio]-1-propanesulfonate, 20 mM DTT; dithiothreitol, 5 mM TCEP; tris(2-carboxyethyl)phosphine hydrochloride) and precipitated with 2-D clean-up kit (Amersham Biosciences) to remove substances interfering with isoelectric focusing (IEF) such as detergents, salts, lipids, phenolic and nucleic acids. IEF was carried out using IPTGhor IEF system (Amersham Biosciences) for the first dimension as recommended by the manufacturer. After first dimension, the strip was cut 5 cm from (+) terminal and 7 cm from (−) terminal. The cut strip was linked with agarose onto 12% SDS-PAGE containing 20 μg/ml dsDNA calf thymus DNA (Calbiochem). Markers and proteins were prepared according to the manufacturer's instructions on Whatman paper and run alongside the cut strip. After electrophoresis, the gel was rinsed with water, and placed into the gel incubation buffer (20 mM Tris-HCl, pH 9.08, 20 mM MgCl₂). Two hours later, the gel was rinsed again and incubated in the gel incubation buffer overnight at 37° C. Then, the gel was transferred into fresh gel incubation buffer and incubated at 37° C. for 1 h containing 1 μg/ml of ethidium bromide. The gel was placed under a 254 nm wave UV light box and photographed. Degradation of DNA by nuclease appeared as dark areas on a brightly staining background of DNA. After gel stained with ethidium bromide, gel was restained with Sypro Ruby (Invitrogen) and scanned with the ProXpress scanner (Perkin Elmer). Spot of interest was excised from gel using a ProXcision Spot cutter (Perkin Elmer), conserved in 1% acetic acid and submitted to trypsin digestion before tandem mass spectrometry analysis. Trypsin fragmentation results were analyzed using Mascot software (Matrix Science) for peptide identification.

### 1.5 Sequence Analysis

The nucleotide sequence obtained was analyzed by NCBI ORF Finder tool (www.ncbi.nlm.nih.gov/gorf/gorf.html). The signal peptide was predicted using Signalp (www.cbs.dtu.dk/services/Signalp). Promoter region was predicted by BPROM (linux1.softberry.com). Blast (www.ncbi.nlm.nih.gov/BLAST) and PFAM (www.sanger.ac.uk/Software/Pfam) searches were performed to identify putative gene function. Nucleic acid and amino acid sequences were aligned using the ClustalW algorithm in Mega 4 software and visualize in the alignment editor (www.megasoftware.net/index.html).

### 1.6 Sequencing

The 783-bp DNA fragment Nucyep1 6×His-tagged (non-specific nuclease, with peptide signal cutting, without stop codon), encoding Y. enterocolitica subsp. palearctica nuclease, was amplified by PCR using Y. enterocolitica subsp. palearctica crude nucleic acid extracts as template and oligonucleotides primers: (NucF1) 5′-TTAATTATTCATATGTCCGCGCCCAAAACC-3′ (SEQ ID NO.1) and (NucR1) 5′-AATATACTCGAGATCGCATCCAATTGT-3′ (SEQ ID NO.2) containing Nde1 and Xho1 restriction sites at the 5′- and 3′-end, respectively, for subsequent cloning. At the same time, the whole 852 bp DNA fragment Nucyep2 6×His-tagged (without stop codon) was amplified by oligonucleotides primers: (NucF2) 5′-ATATCCATGGCGAAATTTAATCTGATTAAA-3′ (SEQ ID NO.3) and (NucR2) 5′-ATAAAAGGATCCATCGCATCCAATTGT-3′ (SEQ ID NO.4) containing Nco1 and BamH1 restriction sites at the 5′- and 3′-end respectively. PCR mixtures contained primers (NucF1 and NucR1 or NucF2 and NucR2) each at 1.0 μM, 200 μM deoxyribonucleoside triphosphates (Amersham Biosciences,), 10 mM Tris-HCl (pH 9.0), 50 mM KCl, 0.1% Triton X-100, 2.5 mM MgCl₂, 3.3 μg/μl bovine serum albumin (Sigma-Aldrich Canada Ltd.,), and 0.025 U/μL Taq DNA polymerase (Promega,) 5 μl genomic DNA was added to each PCR reaction (45 μl), which was subjected to thermal cycling (3 min at 94° C. followed by 40 cycles of 1 s at 95° C., 1 min at 60° C., and 1 min at 72° C., with a final extension of 7 min at 72° C.) using a PTC-200 DNA Engine thermocycler (Bio-Rad Laboratories,). Amplicons were detected using 1.2% agarose gel electrophoresis with 0.25 μg/mL of ethidium bromide in Tris-borate-EDTA buffer (89 mM Tris, 89 mM boric acid, 2 mM EDTA). Amplicon sizes were verified with a 1 kb DNA ladder used as a molecular weight marker (Invitrogen,). Amplification products were purified using gel electrophoresis (1.2% agarose at 120 V for 1 h), followed by staining with methylene blue (Mallinckrodt Baker,) and DNA was purified with the QIAquick gel extraction kit. Both strands of each amplicon were sequenced using an automated ABI sequencer (Applied Biosystems,) with 5 μM of sequencing primers. Chromatogram assembly and analysis were performed using the Sequencher 3.1 software (Gene Codes Corp.,).

### 1.7 Cloning and Overexpression

The nuclease gene purified PCR products were digested with Nde1 and Xho1 for Nucyep1, Nco1 and BamH1 for Nucyep2, respectively. At the same time the expression vectors PET-24a (+) and PET-24d (+) were digested with Nde1 and Xho1 or Nco1 and BamH1, respectively. The digested PCR products and expression vector were purified with QIAquick PCR purification kit. Nucyep1 and Nucyep2 were ligated into digested PET-24a (+) and PET-24d (+) individually by T4 DNA ligase. Competent cells DH5a:F⁻ 80 dlacZ M15 (lacZYA-argF) U169 recA1 endA1hsdR17(rₖ⁻, mₖ⁺) phoAsupE44⁻thi-1 gyrA96 relA1 and BL21star™ (DE3)plysS: F⁻ompT hsdS_{B}(rB⁻mB⁻)gal dcm me131(DE3)plysS(Cam^{R}) carrying inducible isopropyl-1-thio-β-D-galactopyranoside-inducible T7 RNA polymerase gene (Invitrogen) were grown on Luria Bertani (LB) plates with 50 μg/ml kanamycin (Sigma). Transformation of E. coli cells via electroporation was carried out using the Gene pulser electroprotocols provided by Bio-RAD according to manufacturer instructions. Following electroporation into E. coli DH5a, several plasmids were purified by QIAprep spin Miniprep kit. The plasmids carrying the particular gene fragment were used for sequence examination with standard T7 promoter primer and T7 terminator primer. One of the correct recombinant plasmid was electroporated into E. coli BL21 Star™ (DE3)plysS. All transformants were plated on LB agar with 50 μg/ml kanamycin. An isolated colony was then inoculated into LB medium containing 50 μg/ml kanamycin and grown overnight at 37° C. with shaking. The overnight culture was diluted 1:20 in fresh LB medium containing 50 μg/ml kanamycin and grown with shaking at 37° C. until A₆₀₀ reached 0.6. Then IPTG was added to a final concentration 1 mM and the cell culture was incubated for an additional 3 h at 37° C. and 200 rpm. Different induction conditions were tested (IPTG concentration, temperature, time), but the above condition was the best one.

### 1.8 Purification of Recombinant Nuclease

E. coli BL21star™ (DE3)plysS cells were pelleted by centrifugation at 3 000 g, at 4° C. for 10 min and resuspended in lysis buffer (8 M Urea; 0.1 M NaH₂PO₄; 0.01 M Tris-Cl; pH 8.0) and the His-tagged recombinant protein was isolated from the crude cell extract according to the instructions described in Qiagen's QIAexpressionist handbook. Elution fractions from Ni-NTA agarose column containing the recombinant protein were collected and desalted by Centriprep® Centrifugal Filter Devices with Ultracel® YM-10 (Milipore).

### 1.9 Assay of Nuclease Activity

Standard (20 μl) reaction mixture contained 2 μg of calf thymus dsDNA in activity buffer (20 mM Tris-HCl, 20 mM MgCl₂ at pH 7.24). When required, several concentrations of alternative divalent cations, BaCl₂, CaCl₂, NaCl, MnSO₄, NiSO₄, MgCl₂, CoCl₂, KCl, were tested and the effects of other compounds (0.1, 0.25, 0.5 and 1 mg/ml BSA, 0.05, 0.1, 0.5 and 1 mM dNTPs, 2, 5, 10, 20, 50, 100 mM EDTA, 5, 15, 30, 50% glycerol, 0.1, 0.25, 0.5 and 1% SDS, and 1, 2, 3, 4, 5, 6, 7 and 8 M urea) were investigated. To examine substrate specificity, various substrates were tested: calf thymus dsDNA, calf thymus ssDNA, (ssDNA PCR products [Boissinot et al. 2007, Clin Chem. 53:2020-3]), plasmid DNA PET-24d (+), transcription viral RNA. In all cases, the reaction was started by the addition of 2 μl of nickel column purified enzyme, and terminated by adding 4 μl of agarose gel loading buffer (0.1 M EDTA, pH 8.0, 0.05% bromophenol blue, 50% glycerol). Nuclease activity was evaluated by assessing DNA degradation level on gel electrophoresis (1.2% agarose, with 0.25 μg/ml of ethidium bromide, at 170 V for 30 min, in Tris-borate EDTA buffer).

### Example 2

### Results

### 2.1 Evidence of Heat-Stable Sugar Non-Specific Nuclease in Yersinia Strains

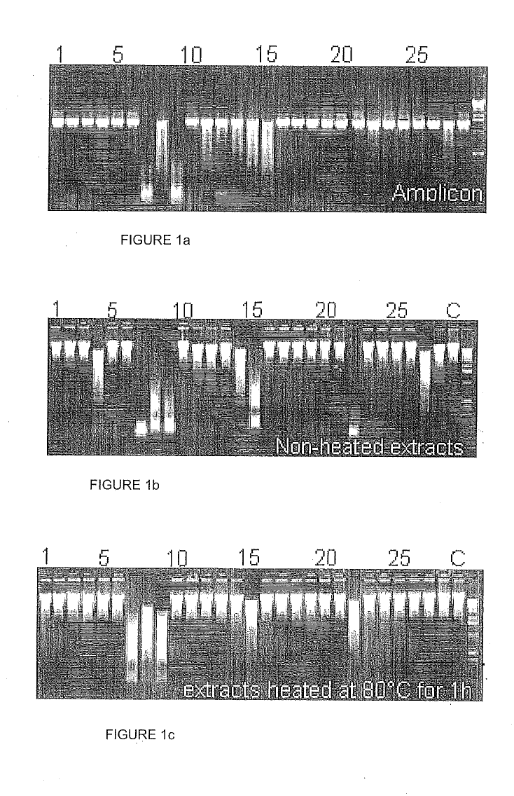

In our laboratory, we initially observed that PCR amplicons prepared with heated (95° C., 5 min) and conserved by freezing DNA crude extracts of Y. enterocolitica subsp. palearctica group I^{[11]} were degraded while stored at 4° C. prior to gel analysis. Therefore, the Y. enterocolitica subsp. palearctica group I DNA crude extracts contained heat-stable nucleases, resistant to freezing, active at 4° C. in standard PCR reaction buffer. The presence of thermostable nuclease activity in representative Yersinia, genetically related Enterobacteriaceae as well as one Staphylococcus aureus strains was evaluated. After PCR reaction using DNA crude extracts, 16S rDNA amplicons were incubated at room temperature (22° C.) for 24 h, then electrophoresed on 1.2% agarose gel. Amplicons' degradation phenomenon was showed on FIG. 1a. At the same time, nuclease activity from heated (80° C., 1 h) crude extracts (FIG. 1c) and non-heated crude extracts (FIG. 1b) was tested for nuclease enzyme activity using calf thymus DNA degradation monitored by agarose gel electrophoresis to evaluate size reduction of DNA.

TABLE 1
Nuclease activity in FIGS. 1a, 1b and 1c

| | Strains | FIG. 1a | FIG. 1b | FIG. 1c |
|---|---|---|---|---|
| 1 | Yersinia bercovieri ATCC 43970^{T} (CCRI-763) | − | − | − |
| 2 | Yersinia aleksiciae LMG 22254^{T} (CCRI-15962) | − | − | − |
| 3 | Yersinia mollaretii ATCC 43696^{T} (CCRI-768) | − | − | − |
| 4 | Yersinia frederiksenii ATCC 33641^{T} (CCRI-765) | + | + | − |
| 5 | Yersinia intermedia ATCC 29909^{T} (CCRI-766) | − | − | − |
| 6 | Yersinia aldovae ATCC 35236^{T} (CCRI-14535) | − | − | − |
| 7 | Yersinia enterocolitica subsp. palearctica group I CCRI-10035 | +++ | +++ | ++ |
| 8 | Yersinia enterocolitica subsp. palearctica group I CCUG 4586 (CCRI-14538) | ++ | ++ | ++ |
| 9 | Yersinia enterocolitica subsp. palearctica group I CCUG 21476 (CCRI-14540) | +++ | +++ | ++ |
| 10 | Yersinia kristensenii ATCC 33638^{T} (CCRI-767) | − | − | − |
| 11 | Yersinia enterocolitica subsp. enterocolitica ATCC 9610^{T} (CCRI-764) | + | + | − |
| 12 | Yersinia enterocolitica subsp. enterocolitica ATCC 27729 (CCRI-2161) | + | + | − |
| 13 | Yersinia enterocolitica subsp. enterocolitica CCUG 8238 (CCRI-14541) | + | + | − |
| 14 | Yersinia enterocolitica subsp. palearctica group II CCUG 18381 (CCRI-14539) | ++ | + | − |
| 15 | Yersinia enterocolitica subsp. palearctica group II CCUG 31436 (CCRI-14542) | ++ | ++ | + |
| 16 | Yersinia frederiksenii ATCC 29912 (CCRI-14530) | − | − | − |
| 17 | Yersinia rohdei ATCC 43380^{T} (CCRI-771) | − | − | − |
| 18 | Yersinia ruckeri CCRI-10643 | − | − | − |
| 19 | Yersinia pseudotuberculosis ATCC 29833^{T} (CCRI-769) | − | − | − |
| 20 | Hafnia alvei ATCC 13337^{T} (CCRI-545) | − | − | − |
| 21 | Obesumbacterium proteus ATCC 12841^{T} (CCRI-642) | − | − | − |
| 22 | Serratia marcescens ATCC 13880^{T} (CCRI-736) | + | +++ | + |
| 23 | Yokenella regensburgei ATCC 35313^{T} (CCRI-574) | − | − | − |
| 24 | Klebsiella pneumoniae ATCC 13883^{T} (CCRI-566) | − | − | − |
| 25 | E. coli ATCC 11775^{T} (CCRI-467) | − | − | − |
| 26 | Staphylococcus aureus ATCC 43300 (CCRI-175) | − | − | − |
| 27 | Salmonella enterica subsp. enterica ATCC 13076^{T} (CCRI-722) | + | + | − |
| 28 | Salmonella Typhimurium ATCC 14028 (CCRI-717) | − | − | − |
| C | Negative control-Calf thymus DNA extract | − | − | − |
| Right | Molecular weight marker | NA | NA | NA |
| lane | | | | |

Legend:
−: no DNA degradation observed
+: weak nuclease activity observed
++: intermediate nuclease activity observed
+++: strong nuclease activity observed
NA: not applicable

As a result, PCR products' degradation phenomenon was observed in Y. enterocolitica subsp. palearctica, Y. enterocolitica subsp. enterocolitica, S. marcescens, Salmonella choleraesuis, and Y. frederiksenii. Heat-stable nuclease activity was only detected in Y. enterocolitica subsp. palearctica CCRI-10035, CCUG 4586 (CCRI-14538), CCUG 21476 (CCRI-14540) and CCUG 31436 (CCRI-14542), and S. marcescens CCRI-736. It is interesting that heat-stable nuclease activity can be detected in Y. enterocolitica subsp. palearctica, but can not be detected in Y. enterocolitica subsp. enterocolitica. Among the ten (10) Y. enterocolitica subsp. palearctica strains studied (5 are listed in Table 1: 4 positives and 1 negative), only the strain CCUG 18381 (CCRI-14539) did not show heat-stable nuclease activity.

Heat-stable nuclease activity level is quite different from one strain to another, among all the ten tested Y. enterocolitica subsp. palearctica strains, the strain CCRI-10035 presented the highest heat-stable nuclease activity level. DNA degradation phenomenon can be observed in a wide range of incubation temperature, from 0 to 80° C. The heated crude extracts of Y. enterocolitica subsp. palearctica CCRI-10035 can also degrade ssDNA, RNA and circular plasmid DNA. The degradation phenomenon can be prevented by keeping products in −20° C., addition of EDTA or agarose gel loading buffer, or a treatment with proteinase K (data not shown).

2.2 Partial Purification of Crude Extracts from Y. enterocolitica subsp. palearctica CCRI-10035 and Active Two-Dimensional Gel Electrophoresis

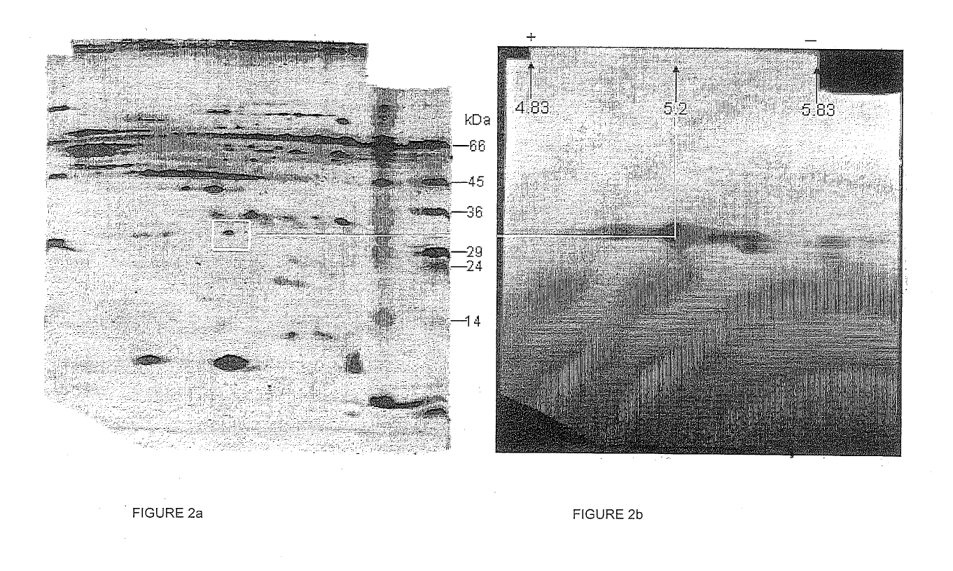

Crude extracts were heated at 80° C. for 1 h, cooled down, and centrifuged for 20 min at 10 000 g, 4° C. The supernatant was loaded on a heparin affinity column, the elution fractions containing nuclease activity was collected and desalted by dialysis in 20 mM Tris-Cl pH 9.08 buffer with 20 mM MgCl₂. Sample was concentrated to 1.5 mg/ml by Microcon Centrifugal filter devices ultracel YM-10 (Millipore). Then the above protein sample was diluted into T8 buffer following active two-dimensional gel electrophoresis. Photos of gels stained with ethidium bromide and Sypro Ruby are shown on FIG. 2a and FIG. 2b, respectively.

The spot with heat-stable nuclease activity has a molecular weight of approximately 32 kDa, and a μl of about 5.2. The marked square spot was cut and subjected to tandem mass spectrometry analysis. Candidate peptides were obtained. Primers were designed at highly conserved regions according to amino acid multiple alignment results (data not shown). After PCR reaction (DNA template is the crude extracts of Y. enterocolitica subsp. palearctica CCRI-10035), nucleotides sequences were obtained. The blast results on NCBI showed all the nucleotides sequences were almost the same as nucleotides sequences in genome project of Y. enterocolitica subsp. enterocolitica 8081. When we investigated heat-stable nuclease activity, we found heat-stable nuclease activity was present in Y. enterocolitica subsp. palearctica, but not in Y. enterocolitica subsp. enterocolitica.

2.3 Genomic Analysis of Candidate Nucleases from Y. enterocolitica subsp. enterocolitica 8081

The candidate nuclease could not be precisely identified from proteomic data analyses, so we tried the genomic analysis of nucleases from the genome project of Y. enterocolitica subsp. enterocolitica. We tried candidate nucleases that had close molecular weight and pl to proteomic data of Y. enterocolitica subsp. palearctica (32 kDa, pl 5.2). Primers were designed from highly conserved amino acid region by multiple alignments. Results are shown on Table 2. From Table 2, we found that there were differences of YE2923's deduced amino acid between Y. enterocolitica subsp. palearctica and Y. enterocolitica subsp. enterocolitica. Furthermore, the pl was lower in Y. enterocolitica subsp. palearctica, which was closer to the proteomic data. The full length of YE2923 is 283 amino acids. However, we obtained only 130 amino acids of Y. enterocolitica subsp. palearctica CCRI-10035. We designed new primers to get the entire deduced amino acid sequence in Y. enterocolitica subsp. palearctica. From the other Yersinia genome project, we found that, for the protein YE2923 there are differences between Y. enterocolitica subsp. enterocolitica and other Yersinia genome project, but there are less differences for the neighbor genes: YE2923, YE2924 and YE2922. Forward primers were designed at the end of YE2922 (putative protease) according to nucleotides multiple alignment, and reverse primer at the beginning of YE2924 (phosphoglycerate) according to the nucleotide sequence multiple alignment. We could obtain amplicons of nuclease for all the Y. enterocolitica subsp. palearctica strains except one strain CCRI-14539 which has no heat-stable nuclease.

TABLE 2
Putative nuclease from genomic analysis of Y. enterocolitica subsp.
enterocolitica.

| | | Calculated | Amino acid blast results |
|---|---|---|---|
| | | MW kDa/ | Y. enterocolitica subsp. palearctica vs |
| Gene name | Protein name | pI | Y. enterocolitica subsp. enterocolitica |
| YE1642 | Putative | 29.8/5.23 | Identities = 151/151 (100%), Positives = |
| | deoxyribonuclease | | 151/151 (100%), Gaps = 0/151 (0%) |
| YE1790 | Putative | 25/5.87 | Identities = 197/198 (99%), Positives = |
| | exodeoxyribonuclease | | 198/198 (100%), Gaps = 0/198 |
| YE2254 | xthA exonuclease III | 30.7/5.49 | Identities = 210/211 (99%), Positives = |
| | | | 211/211 (100%), Gaps = 0/211 (0%) |
| YE2355 | exodeoxyribonuclease | 25/5.59 | Identities = 173/173 (100%), Positives = |
| | | | 173/173 (100%), Gaps = 0/173 (0%) |
| YE2923 | DNA/RNA non- | 30.5/7.58 | Identities = 120/130 (92%), Positives = |
| | specific | | 123/130 (94%), Gaps = 0/130 (0%) |
| | endonuclease | | |

2.4 Nucleotide Sequence Analysis of Nuclease from Y. enterocolitica subsp. palearctica CCRI-10035

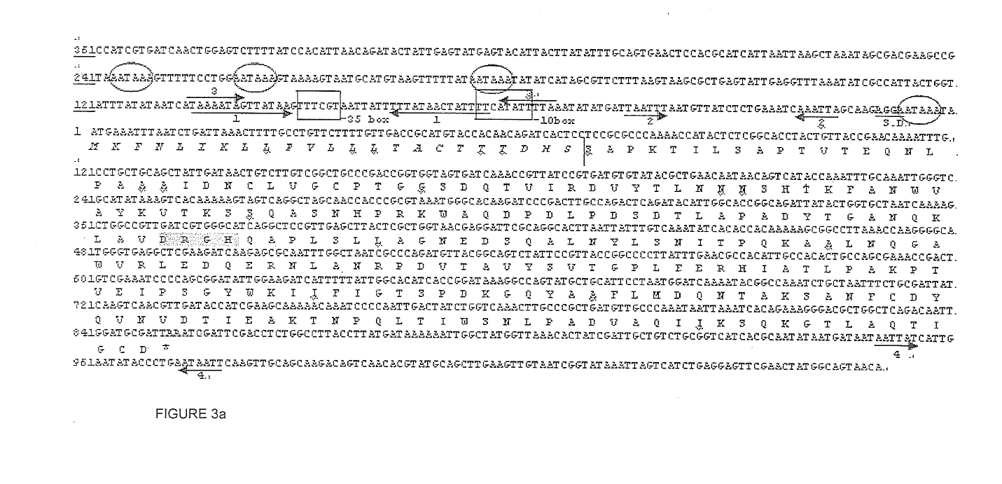

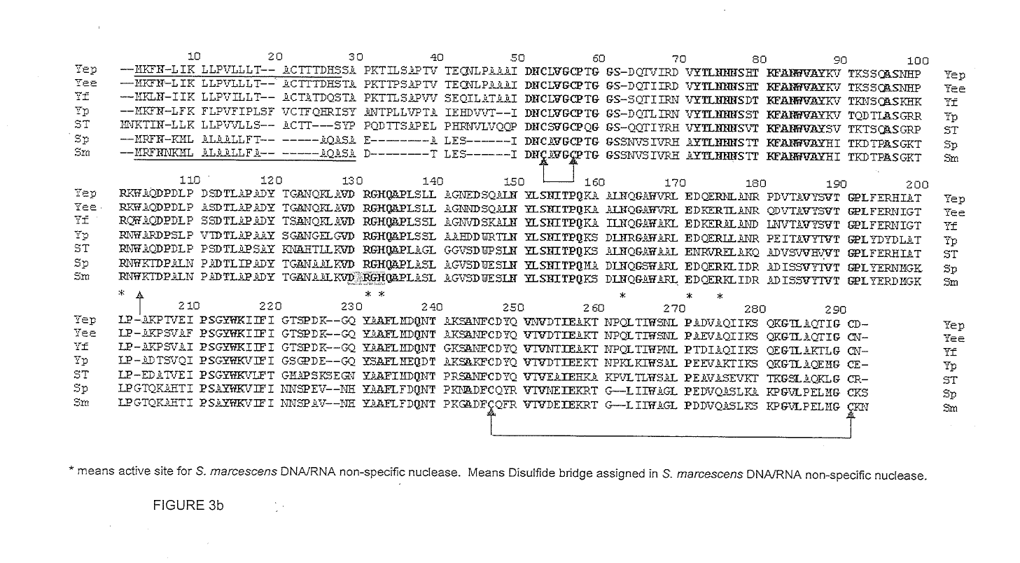

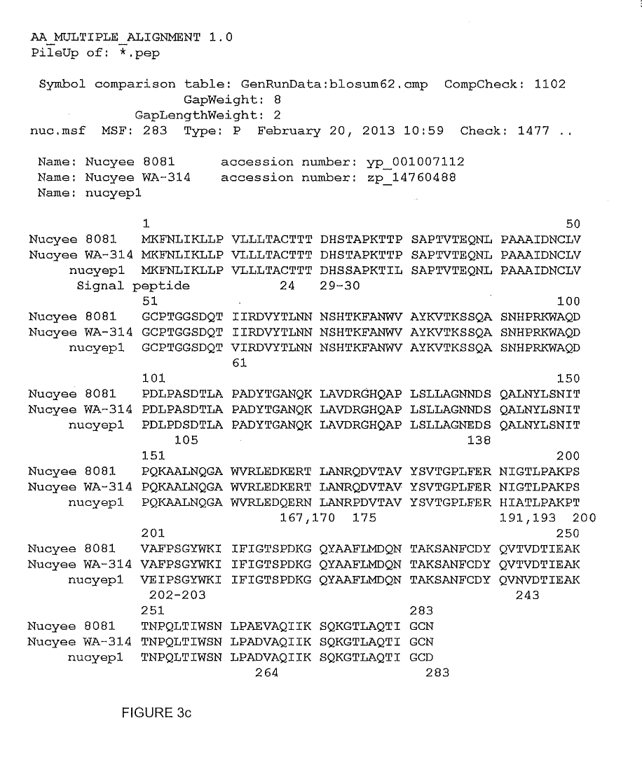

When primers were designed in the neighbor gene YE2923 (Y. enterocolitica subsp. enterocolitica), the entire nucleotide sequence of Y. enterocolitica subsp. palearctica was obtained. Analysis of the DNA sequence across the region that coded for nuclease identified an open reading frame (ORF), designated Nucyep, of 852 bp coding for a 283-amino-acid protein of 30.7 KDa, pl 5.95 (FIG. 3a). Shine-Dalgarno-like sequence (AGGA) was found 7 bp upstream of the ATG start codon of this nuclease. A putative promoter (−10 and −35 regions) predicted to be located 59 bp upstream of the initiation codon ATG, where was AT rich with several potential TATA box and possible ribosomal binding sites. Repeated AATAAA sequences were found upstream of Nuc, which is supposed to enhance promoter activity. When we look at other Yersinia strains promoter region, we could not see the same repeated sequences. We believe that transcription termination most probably occurs at an inverted repeat sequence downstream. For the deduced amino acid sequence, the first 23 amino acids were predicted to be peptide signal. Amino acid pairwise alignment was also carried out with Pir software (pir.georgetown.edu/pirwww/search/pairwise.shtml). Results show there are 17 different amino acids between Y. enterocolitica subsp. palearctica CCRI-10035 (Yep) and Y. enterocolitica subsp. enterocolitica 8081 (Yee), and that they share 93% identity. There are 47 different amino acids between Y. enterocolitica subsp. palearctica and Y. frederiksenii ATCC33641, and they share 83% identity. Other strains share less homology with Y. enterocolitica subsp. palearctica. Amino acid sequences multiple alignment results are shown in FIG. 3b. Alignment of Nucyep1 (SEQ ID NO. 8) with the closest sequences from Yersinia enterocolitica subsp. enterocolitica (these nucleases don't exhibit the same thermal and pH properties) shows 94% identity when including the signal peptide (267/283 with Nucyee WA-314 and 266/283 with Nucyee 8081) (FIG. 3c). Excluding the signal peptide, there is ˜94% identity (244/260) with Nucyee WA-314 qnd ˜93% identity (243/260) with Nucyee 8081.

2.5 Cloning and Expression of the Nuclease Gene from Y. enterocolitica subsp. palearctica

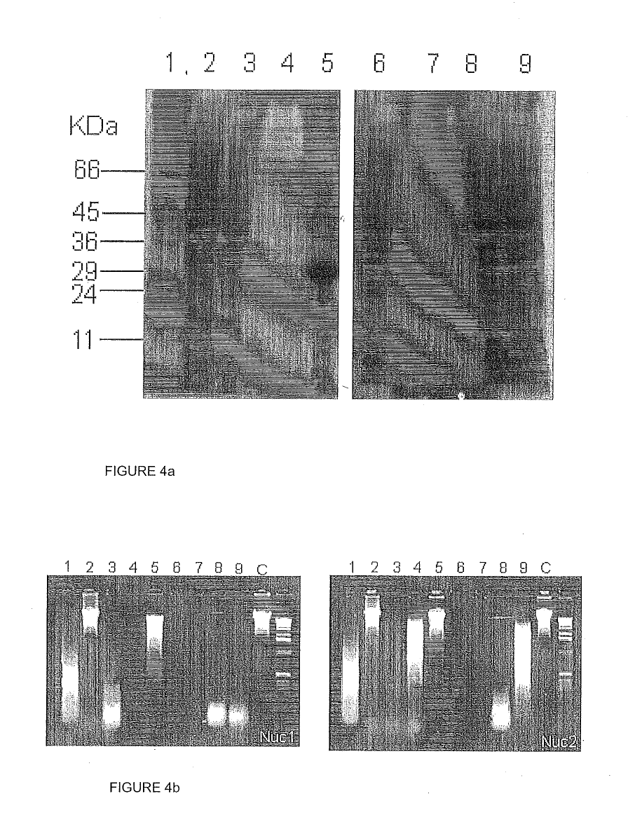

Prior to this study, expression of recombinant non-specific nuclease from Yersinia strains had never been reported. The particular Nucyep1 fragment (without peptide signal and stop codon, with C-terminal 6×His-tag; SEQ ID NO. 8) was first cloned into PET-24a (+) expression vector, then transformed into E. coli DH5a by electroporation and plated onto LB agar overnight. Thirteen (13) colonies were picked and plasmids were purified by QIAprep spin Miniprep kit. Among the 13 colonies, only 5 colonies carried the particular gene fragment. The plasmids carrying the particular gene fragment were used for sequence examination with standard T7 promoter primer and T7 terminator primer. As a result, only one colony had the correct insert. The recombinant plasmid with the correct nucleotide sequence was electroporated into E. coli BL21star™ (DE3)plysS. The particular Nucyep2 fragment (with peptide signal, without stop codon, with C-terminal 6×His-tag; SEQ ID NO. 10) was cloned into pET-24d(+) expression vector, then transformed into E. coli DH5a by electroporation, plated onto LB agar overnight. Seven (7) colonies were picked and plasmids were purified by QIAprep spin Miniprep kit. Among these 7 colonies, 5 colonies carried the particular gene fragment. The plasmids carrying the particular gene fragment were used for sequence examination with standard T7 promoter primer and T7 terminator primer. And as a result, two colonies had the correct inserts. One of those was electroporated into E. coli BL21star™ (DE3)plysS. After induction with IPTG at 37° C., the induced and non-induced recombinant bacteria and control bacteria (just vector, no insert) were analyzed by SDS-PAGE (FIG. 4a) and the nuclease activity was also detected (FIG. 4b).

For Nucyep1, a very strong band corresponding to the fusion protein was observed in the induced recombinant bacteria (FIG. 4a, lane 5). However, no obvious strong band was observed for Nucyep2 in the induced recombinant bacteria (FIG. 4a, lane 6). There was nuclease activity detected in the induced recombinant bacteria and weak nuclease activity was detected in the non-induced recombinant bacteria, whereas no nuclease activity was detected in the control (no insert, just vector) for both Nucyep1 and Nucyep2. These experiments showed that Nucyep1 and Nucyep2 were expressed.

### 2.6 Purification of Recombinant Nuclease

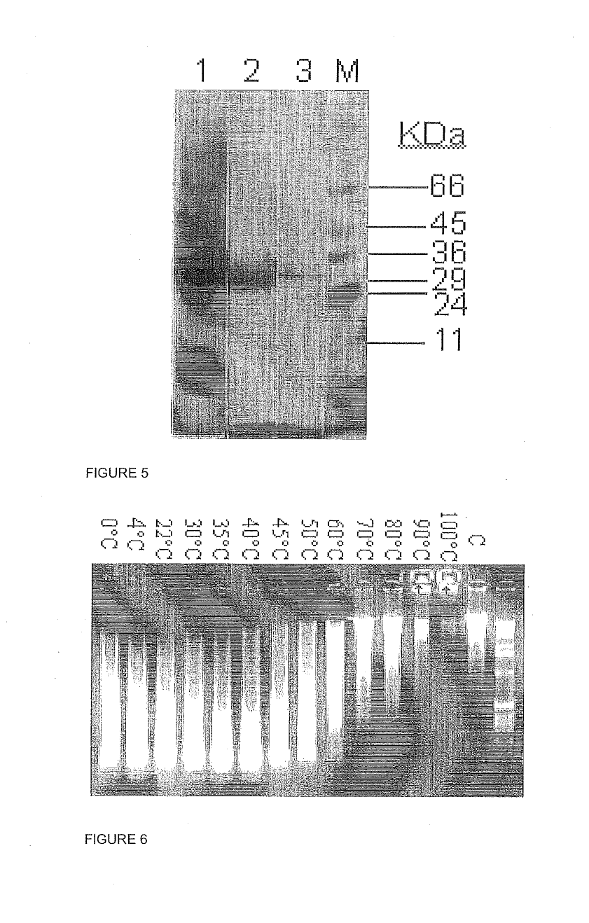

Most of the recombinant protein for Nucyep1 was found in the cytoplasm (data not shown). Cell lysis by French pressure and sonication was carried out, but whatever condition applied, most of the activity still was found in the precipitate, whereas about 1/10 of the nuclease activity remained in the supernatant. We therefore believed that most of the recombinant protein was present in inclusion bodies of E. coli. Inclusion bodies could be seen in induced E. coli by microscopy (data not shown). The supernatant of Nucyep1 was then purified by Ni-NTA spin kit under native condition according to the protocol provided by Qiagen, but the purification efficiency was low, since no purified band could be seen on SDS-PAGE, and the corresponding band was very slim (which means the quantity of recombinant protein was very low). We then tried to purify the recombinant nuclease under denaturing conditions. This time the protocol worked well. After purification, there was still a strong band that could be seen on SDS-PAGE (FIG. 5, lane 2). Protein from lane 2 was loaded onto Sephadex G-100, and one quite pure band was seen on SDS-PAGE (lane 3), however, the recovery was low. For the following experiments, we checked heat stability of the protein after purification on Sephadex G-100. Other experiments were performed on the protein purified with Ni-NTA column. Nucyep2 could not be purified because the quantity was too small.

### 2.7 Optimum Temperature and Heat Stability

The activity of nuclease toward dsDNA was examined within the temperature range 0 to 100° C., and optimal activity was found at 35-40° C. When the substrate was incubated at 90 or 100° C. for 10 min, the DNA aggregated, so we can't be sure if the recombinant Nucyep1 works at 90 and 100° C. However, we do know that this recombinant Nucyep1 works at 0° C., which means it can be used for purification of recombinant proteins that are not stable at room temperature whereas other nucleases would have very low to no activity in such conditions. When purified Nucyep1 was heated at 80° C. for 1 h and 100° C. for 20 min, there was still more than 5% nuclease activity left (data not shown).

### 2.8 Optimum pH and pH Stability

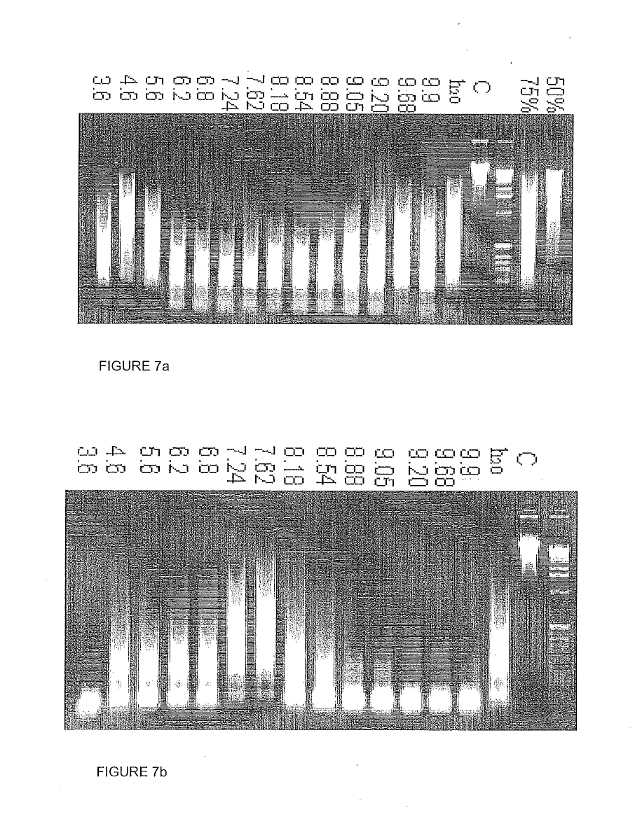

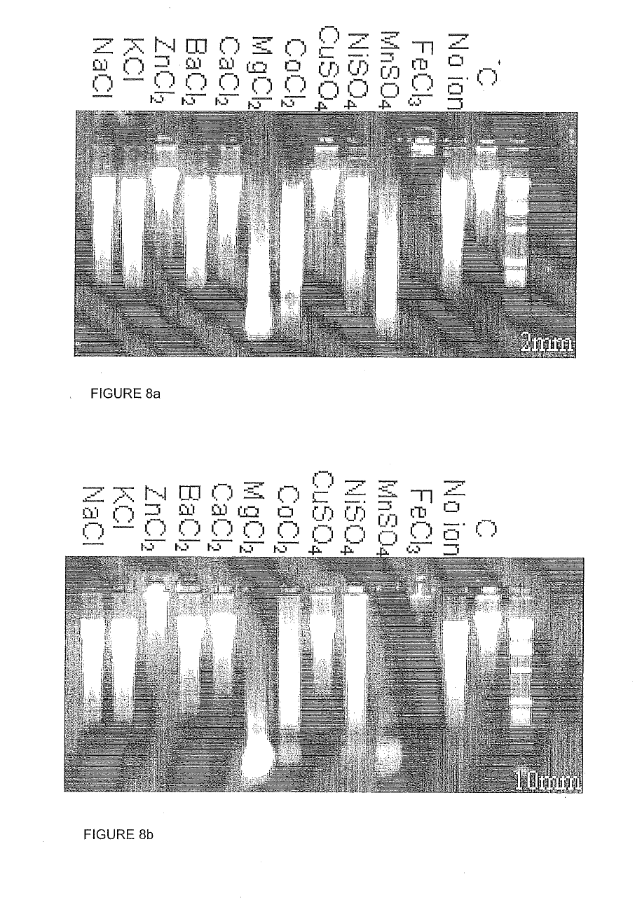

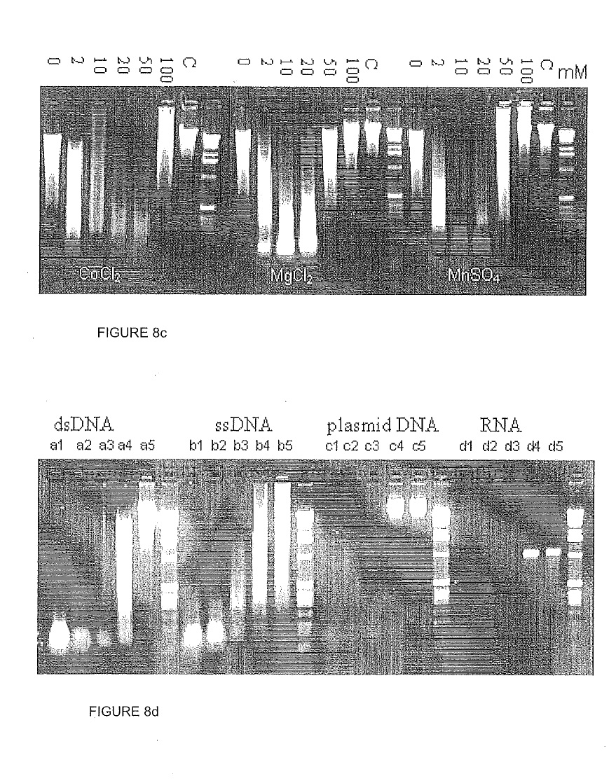

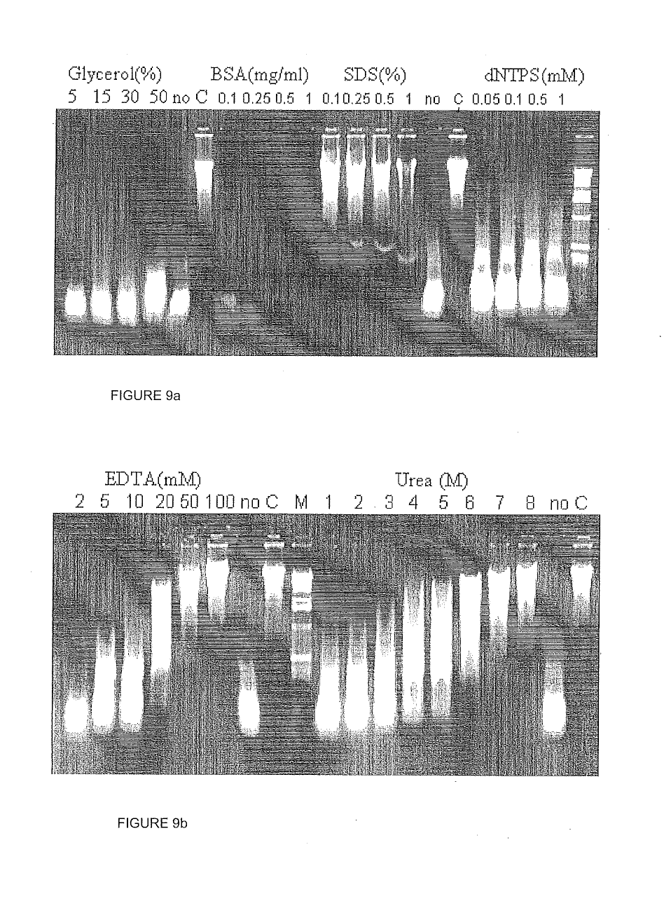

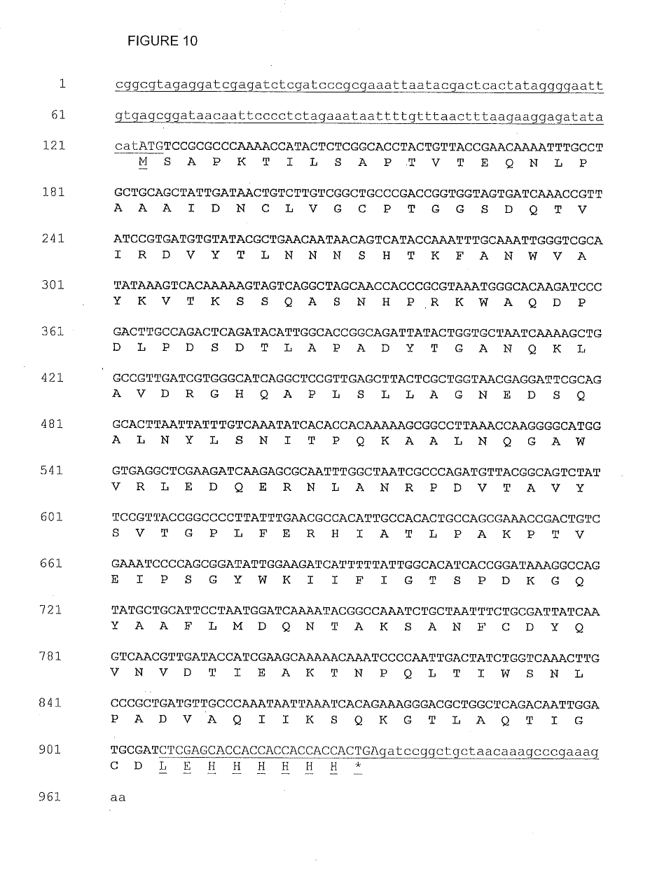

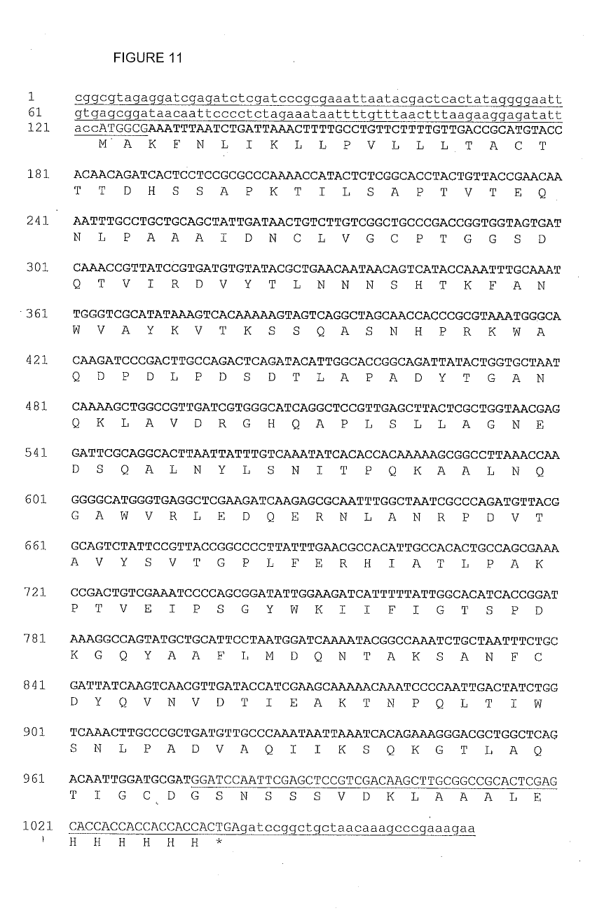

As shown in FIG. 7a, the optimum pH of the purified recombinant Nucyep1 was between 7.24 and 7.62. However, the enzyme is active between pH3.6-9.9, and the nuclease retained greater than 75% of its activity at pH 7.24 which means it can be used to decontaminate enzymatic amplification reactions and recombinant proteins without the need to modify the pH value which could be detrimental to the structure or activity of some proteins. Purified Nucyep1 was incubated at room temperature with 20 mM MgCl₂ in 20 mM Tris-HCl at different pH values for 1 h. The residual activity was checked by standard enzyme assay (FIG. 7b).

### 2.9 Metal Ion Requirements and Substrate Specificity

Activation by a wide variety of metal ions was tested by visualizing the degradation of dsDNA on agarose gels. When FeCl₃ added to the substrate, DNA was precipitated because it precipitated. When the metal ion concentration was adjusted to 2 mM, divalent Mg²⁺ was the best activator. When the metal ion concentration was increased to 10 mM, divalent Mg²⁺ and Mn²⁺ were the best two activators. The nuclease activity could be completely inhibited by 2 mM ZnCl₂, 10 mM CaCl₂, 2 mM CuSO₄. The nuclease activity could be partly inhibited by 10 mM BaCl₂. Two (2) mM and 10 mM NaCl, KCl, CoCl₂, NiSO₄ did not have obvious effects on the nuclease activity, so higher concentration were examined as well as for Mg²⁺ and Mn²⁺. 100 mM NaCl, KCl, NiSO₄ still had no effect on the nuclease activity (data not shown). Friedhoff et al. reported Ni²⁺ can substitute Mg²⁺ to activate the extracellular S. marcescens endonuclease^{[24]}. For Co²⁺, concentrations at 20 mM or 50 mM greatly increased the nuclease activity, whereas for Mg²⁺, the optimal concentration to activate the nuclease activity was reached at 10 mM. When Mg²⁺ concentration reached 50 mM, the effect on enzyme activity was the same as with no metal ion; whereas, when its concentration was increased to 100 mM, the enzyme still had nuclease activity, but notably diminished. For Mn²⁺, the optimal concentration was found to be between 10-20 mM; 50 mM could activate the nuclease activity, but 100 mM did inhibit it. Although Mg²⁺ ions are the preferred ions of most enzymes dealing with nucleic acids, Mn²⁺ is the best activator for our recombinant Nucyep1.

Purified recombinant Nucyep1 showed high nuclease activity on dsDNA, ssDNA, plasmid DNA pET-24a(+) (closed circular double stranded) and RNA with the addition of 20 mM Mg²⁺, Mn²⁺, Co²⁺ individually. For dsDNA and ssDNA, Nucyep1 showed nuclease activity without addition of metal ions. But for plasmid DNA and RNA, Nucyep1 showed an obligate requirement of divalent Mg²⁺, Mn²⁺, Co²⁺. From what we observed, we can now say the recombinant Nucyep1 is a non-specific nuclease. And from its specificity, Nucyep1 could be useful for removing DNA impurities during RNA isolation procedures.

### 2.10 Chemical Compounds Effect

Glycerol concentration from 5%-30% have no effect on nuclease activity, whereas 50% glycerol may show a small inhibition. This recombinant Nucyep1 can therefore be stored in the presence of glycerol. BSA concentrations from 0.1 to 1 mg/ml greatly increased the nuclease activity; maybe because BSA can stabilize the Nucyep1 structure. For SDS, even as little as 0.1% could completely inhibit the nuclease activity. When SDS concentration reached 1%, it however denatured the DNA substrate. dNTPs have little inhibition on the nuclease activity. However, too much dNTPs in the solution may reduce the reaction speed.

The influence of urea concentration (1 to 8M) on the nuclease activity was examined. Even a concentration as low as 1 M urea had an effect on the nuclease activity. With an increased urea concentration, we observed more inhibitory effect. Finally, when the urea concentration was lower than 8 M, the enzyme still had some nuclease activity.

EDTA concentration of 2 mM did not affect the nuclease activity. EDTA concentrations from 5 to 10 mM had slight inhibition effect. With the increase of EDTA concentration, more obvious inhibition phenomenon can be observed. When EDTA concentration reached 100 mM, the nuclease activity was totally inhibited.

### Example 3

### Discussion

Y. enterocolitica is an important gastrointestinal pathogen that can cause a range of human diseases, e.g. mild diarrhea, mesenteric lymphadenitis, and septicemia. After PCR products degradation phenomenon was observed, our hypothesis was that there was heat-stable nuclease in Yersinia strains. However, among all the Yersinia strains we tested, only Y. enterocolitica subsp. palearctica showed the presence of a heat-stable nuclease.

A primary structure analysis showed that Nucyep was homologous to S. marcescens DNA/RNA non-specific endonuclease, which hydrolyzes RNA as well as ssDNA and dsDNA. Analysis using SMART and Signalp 3.0 software, indicated that Nucyep contains a signal peptide (the first 23 amino acid), that may be important for nuclease secretion and domains homologous to the nuclease of Y. enterocolitica subsp. palearctica. Two pairs of primers were designed to obtain the Nucyep domain with two restriction sites for cloning. The first one was named Nucyep1, which is without peptide signal and stop codon, with Nde1 and Xho1 restriction sites inserted, and 6×His-tagged. The other, named Nucyep2, contained the whole Nucyep domain sequence, with Nco1 and BamH1 restriction sites inserted, 6×His-tagged, without stop codon. Nucyep1 was cloned into pET-24a, and Nucyep2 was cloned into pET-24d, both of them transformed into E. coli BL21star™ (DE3)plysS, and induced by 1 mM IPTG. As a result, recombinant Nucyep1 showed a strong band on SDS-PAGE, but, surprisingly, Nucyep2 did not show an obvious band on SDS-PAGE. We then purified Nucyep1 through one step purification procedure using Ni-NTA column.

The ability of Nucyep1 to degrade covalently-closed-circular DNA indicates that it can act as an endonuclease although this does not preclude that the enzyme might also act as an exonuclease. We were unable to detect any specificity in the cutting sites. Nucyep1 is active in a broad pH range from 3.6 to 9.9, and its optimum pH is between 7.24 and 7.62. Nucyep1 is also active at a broad range of temperature from 0 to 80° C., and its optimum temperature is between 35 and 40° C., which is similar with other known non-specific nucleases^{[24, 27, 28, 29]}.

Nucyep1 is heat-stable: it is active after heating at 80° C. for 1 h or 100° C. for 20 min. A possible explanation for its thermostability can be the presence of -high amounts of hydrophobic amino acids that stabilize via mutual interactions the enzyme's active conformation. In contrast to most nucleases, Nucyep1 does not need cations for its action on linear dsDNA and ssDNA, although Mg²⁺, Co²⁺, Mn²⁺ can greatly stimulate its activity. Metal ions may have played a role in stabilizing the enzyme structure. However, it could not be ruled out whether the Nucyep1 was co-purified with some tightly bound metal ion, as was the case with some other nucleases.

Nucyep1 shares 65% homology in amino acid content from S. marcescens. We suspect that the three dimensional structure of Nucyep1 is very similar to that of Nuc from S. marcescens. If they are similar, then two disulfide bridges, cys (53)-cys (57) and cys (247)-cys (291) probably form (numbering according to FIG. 3b). Ball et al^{[34]} tested the role of the two disulfide bonds in the S. marcescens by site directed mutagenesis, and found these two disulfide bonds were essential for nuclease activity, although slight residual activity remained. More precise studies are necessary to obtain exact or probable information concerning the S—S bridge. Genetic approaches to identify the catalytic domain are in progress.

The wide range of action of Nucyep1 could make this enzyme very useful for biotechnological applications: the nucleases currently used to reduce the viscosity of crude cell extracts usually require Mg²⁺ for their action, which also activates proteinases. The heat stability of Nucyep1 would facilitate the applications of nuclease in molecular biology researches and industry at high temperatures, such as the determination of nucleic structure, the removal of nucleic acids during protein purification and the use as antiviral agents.

### REFERENCES

[4] P. Friedhoff, B. Kolmes, O. Gimadutdinow, W. Wende, K. L. Krause, A. Pingoud, Analysis of the mechanism of the Serratia nuclease using site-directed mutagenesis Nucleic Acids Res. 24(1996) 2632-2639.
[5] Y. C. Chen, G. L. Shipley, T. K. Ball, M. J. Benedik, Regulatory mutants and transcriptional control of the Serratia marcescens extracellular nuclease gene, Mol. Microbiol. 6(1992) 643-651.
[6] T. K. Ball, C. R. Wasmuth, S. C. Braunagel, M. J. Benedik, Expression of Serratia marcescens extracellular proteins requires recA, J. Bacteriol. 172(1990) 342-349.
[7] D. M. Mitchell, T. Jack, A. Mary, J. B. Michael, L. K. Kurt, 2.1 Å structure of Serratia endonuclease suggests a mechanism for binding to double-stranded DNA Nat. Struct. Biol. 1(1994) 461-468.
[8] Merck KgaA Darmstadt, Germany, BENZONASE Brochure. Code. No. W220911, 1999.
[9] C. Korn, G. Meiss, F. U. Gast, O. Gimadutdinow, C. Urbanke, A. Pingoud, Genetic engineering of Escherichia coli to produce a 1:1 complex of the Anabaena sp. PCC 7120 nuclease NucA and its inhibitor NuiA, Gene 253 (2000) 221-229.
[10] E. J. Bottone, Yersinia enterocolitica: overview and epidemiologic correlates, Microbes infect. 1 (1999) 323-333.
[11] Isabel S, Leblanc E, Boissinot M Boudrea D K Grondin M Picard F J Martel E A Parham N J Chain P S Bader D E Mulvey M R Bryden L, Roy P H Ouellette M, Bergeron M G, Divergence among Genes Encoding the Elongation Factor Tu of Yersinia Species, J. Bacteriol. 190 (2008) 7548-7558.
[12] H. Nakajima, E. Itoh, M. Arakawa, M. Inoue, T. Mori, H. Watanabe, Degradation of a polymerase chain reaction (PCR) product by heatstable deoxyribonuclease (DNase) produced from Yersinia enterocolitica, Microbiol. Immunol. 38 (1994) 153-156.
[13] J. R. Gibson, R. A. McKee, PCR products generated from unpurified Salmonella DNA are degraded by thermostable nuclease activity, Lett. Appl. Microbiol. 16 (1993) 59-61.
[14] T. Joseph, P. Chaudhuri, B. Sharma, Degradation of PCR products generated from unpurified Salmonella Gallinarum DNAIa Enteritidis Poultry isolates, Indian J. Comp. Microbiol. Immunol. Infect. Dis. 19 (1998) 20-22.
[15] K. Tamura, J. Dudley, M. Nei, S. Kumar, MEGA4: Molecular Evolutionary Genetics Analysis (MEGA) software version 4.0, Mol. Biol. Evol. 24(2007) 1596-1599.
[16] Z. Brnáková, A. Godany, J. Timko, An extracellular endodeoxyribonuclease from Streptomyces aureofaciens, Biochim. Biophys. Acta Gen. Subj. 1721 (2005) 116-123.
[19] F. Picard, C. Menard, Universal method and composition for the rapid lysis of cells for the release of nucleic acids and their detection, US Patent 2004/0076990 A1.
[22] Miller, J. Cai, K. L. Krause, The active site of Serratia endonuclease contains a conserved magnesium-water cluster, J. Mol. Biol. 288(1999) 975-987.
[23] S. V. Shlyapnikov, V. V. Lunin, M. Perbandt, K. M. Polyakov, V. Y. Lunin, V. M. Levdikov, C. Betzel, A. M. Mikhailov, Atomic structure of the Serratia marcescens endonuclease at 1.1 Å resolution and the enzyme reaction mechanism, Acta Crystallogr. D 56(2000) 567-572.
[24] P. Friedhoff, O. Gimadutdinow, A. Pingoud, Identification of catalytically relevant amino acids of the extracellular Serratia marcescens endonuclease by alignment-guided mutagenesis, Nucleic Acids Res. 22 (1994) 3280-3287.
[25] B. Kirsten, K. J. Pia, R. Erik, S. Ib, Purification and characterization of a Serratia marcescens nuclease produced by Escherichia coli, Carlsberg. Res. Comm. 54 (1989) 17-27.
[27] S. Linn, I. R. Lehman, An endonuclease from mitochondria of Neurospora crassa, J. Biol. Chem. 241 (1966) 2694-2699.
[28] A. M. Muro-Pastor, E. Flores, A. Herrero, C. P. Wolk, Identification, genetic analysis and characterization of a sugar-nonspecific nuclease from the cyanobacterium Anabaena sp. PCC 7120, Mol. Microbiol. 6 (1992) 3021-3030.
[29] M. Maeda, N. Taga, Extracellular nuclease produced by a marine bacterium. II. Purification and properties of extracellular nuclease from a marine Vibrio sp., Can. J. Biochem. 22 (1976) 1443-1452.
[30] M. Miertzschke, T. Greiner-Stöffele, The xthA gene product of Archaeoglobus fulgidus is an unspecific DNase, Eur. J. Biochem. 270 (2003) 1838-1849.
[31] A. Stevens, R. J. Hilmoe, Studies on a nuclease from Azotobacter agilis I. Isolation and mode of action, J. Biol. Chem. 235 (1960) 3016-3022.
[32] E. Rangarajan, V. Shankar, Extracellular nuclease from Rhizopus stolonifer: purification and characteristics of single strand preferential deoxyribonuclease activity, Biochim. Biophys. Acta 1473 (1999) 293-304.
[33] G. A. Cordis, P.-J. Goldblatt, M. Deutscher, Purification and characterization of a major endonuclease from rat liver nuclei, Biochemistry 14 (1975) 2596-2603.
[34] T. K. Ball, Y. Suh, M. J. Benedik, Disulfide bonds are required for Serratia marcescens nuclease activity, Nucleic Acids Res. 20 (1992) 4971-4974.
Batzilla, J., Hoper, D., Antonenka, U., Heesemann, J. and Rakin, A. Complete Genome Sequence of Yersinia enterocolitica subsp. Palearctica Serogroup O:3 J. Bacteriol. 193 (8), 2067 (2011)
Boissinot K. et al 2007 Clin Chem. November; 53(11): 2020-3.
Boissinot M. et al 1997, EMBO J. 16:2171-8.
Kunitz M. 1950, J. Gen. Physiol., 33, 349-362).
Neubauer, H., A. Stojanka, H. Andreas, F. Ernst-J.u\ddot{}rgen, and M. Hermann. 2000. Yersina enterocolitica 16S rRNA gene types belong to the same genospecies but form three homology groups. Int. J. Med. Microbiol. 290:61-64. (before 11)

1. A method for degrading DNA or RNA, comprising contacting a DNA or RNA molecule with an effective amount of an isolated protein comprising amino acids starting at about position 20 and ending at about position 261 of SEQ ID NO.8, wherein said protein has nuclease activity at about 4° C. and wherein said contacting is carried out within a range of pH of about 3.6 to about 9.9 under conditions to degrade the DNA or RNA.

2. The method of claim 1, wherein said DNA is selected from the group consisting of a single-stranded DNA (ssDNA) and linear or circular double-stranded DNA (dsDNA).

3. The method of claim 1, wherein said contacting is carried out within a temperature of about 0° C. to about 80° C.

4. A kit for degrading DNA or RNA under heat or extreme pH conditions, said kit comprising at least one isolated protein comprising amino acids starting at about position 20 and ending at about position 261 of SEQ ID NO.8, wherein said protein has nuclease activity at about 4° C., within a range of pH of about 3.6 to about 9.9, in a recipient and instructions on how to use the kit.

5. A kit for degrading DNA or RNA under heat or extreme pH conditions, said kit comprising at least one nucleic acid that encodes at least one isolated protein comprising amino acids starting at about position 20 and ending at about position 261 of SEQ ID NO.8, wherein said protein has nuclease activity at about 4° C., within a range of pH of about 3.6 to about 9.9 in a recipient and instructions on how to produce and use the at least one isolated protein.

6. The kit of claim 5, comprising a plasmid comprising at least one nucleic acid that encodes the at least one isolated protein in a recipient and instructions on how to use said plasmid for producing a protein having nuclease activity, and instructions on how to use said protein for degrading DNA or RNA.

7. The kit of claim 6, comprising a cell that is transformed or transduced with said at least one nucleic acid in a recipient and instructions on how to use said cell for producing a protein having nuclease activity, and instructions on how to use said protein for degrading DNA or RNA.

8. The kit of claim 5, wherein said protein comprises amino acids 2 to 261 of SEQ ID NO.8.

9. A kit for degrading DNA or RNA under heat or extreme pH conditions, said kit comprising at least one nucleic acid that encodes at least one isolated protein comprising an amino acid sequence represented by SEQ ID NO. 6, 8 or 10, wherein the protein, wherein said protein has nuclease activity at about 4° C., within a range of pH of about 3.6 to about 9.9, in a recipient and instructions on how to produce and use the at least one isolated protein.

10. The method of claim 1, wherein said protein is stable at a range of temperatures from about 50° C. to about 100° C.

11. The method of claim 1, wherein said protein has a molecular weight by SDS polyacrylamide gel electrophoresis of about 29 kDa to about 32 kDa.

12. The method of claim 1, wherein said protein has at least 95% identity with the protein isolated protein comprising amino acids starting at about position 20 and ending at about position 261 of SEQ ID NO.8, said protein retaining nuclease activity at about 4° C.

13. The method of claim 1, wherein said protein has at least 95% identity with the protein as defined by SEQ ID NO. 6, 8 or 10.

14. The method of claim 13, wherein said protein is encoded by a nucleic acid sequence which hybridizes to a sequence defined by SEQ ID NO. 5, 7 or 9, under stringent conditions.

15. The method of claim 14, wherein said nucleic acid has a sequence defined by SEQ ID NO. 5, 7 or 9.

16. The method of claim 15, wherein said nucleic acid has a sequence defined by SEQ ID NO. 7.

17. The method of claim 1, wherein said protein is recombinant.
